(19) United States
(12) Patent Application Publication
(10) Pub. No.: US 2026/0337280 A1
(43) Pub. Date: Oct. 29, 2026

(54) METHODS FOR TREATING A BACTERIAL INFECTION

(71) Applicant: BOARD OF SUPERVISORS OF LOUISIANA STATE UNIVERSITY AND AGRICULTURAL AND MECHANICAL COLLEGE, Baton Rouge, LA (US)

(72) Inventor: Monica C. Gestal, Metairie, LA (US)

(21) Appl. No.: 19/114,689

(22) PCT Filed: Sep. 26, 2023
(86) PCT No.: PCT/US2023/033723

§ 371 Date: Mar. 24, 2025

Related U.S. Application Data
(60) Provisional application No. 63/409,890, filed on Sep. 26, 2022.

(51) Int. Cl. C07K 16/28; A61K 39/00; A61P 31/04

(52) U.S. Cl. CPC C07K 16/2866; A61P 31/04; A61K 2039/505; C07K 2317/76

(57) ABSTRACT

Aspects of the invention are drawn to methods for treating or preventing a bacterial infection in a subject. Further aspects of the invention are drawn to methods for sensitizing an antibiotic resistant bacterial infection to an antibiotic.

This application claims priority from U.S. Provisional Application No. 63/409,890 filed on Sep. 26, 2022, the entire contents of which is hereby incorporated by referenced.

### GOVERNMENT SUPPORT

This invention was made with government support under P20 GM134974 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

Aspects of the invention are drawn to methods for treating or preventing a bacterial infection in a subject. Further aspects of the invention are drawn to methods for sensitizing an antibiotic resistant bacterial infection to an antibiotic.

### BACKGROUND OF THE INVENTION

Antibiotic resistance is one of the major threats of humankind and W.H.O. estimates a rate of 10 million deaths annually by 2050. Although, there is a current trend to revisit old remedies, such as old antibiotics; there is the imperative need to provide new therapeutical targets focus on improving host immune responses, rather than killing the bacterial threat.

### SUMMARY OF THE INVENTION

An aspect of the invention is directed to a method of treating or preventing and/or treating a bacterial infection in a subject. In embodiments, the method comprises administering to a subject an IL-1RA inhibitor.

In embodiments, the bacteria comprises Bordetella spp. For example, the bacteria comprises B. pertussis, B. parapertussis, B. bronchiseptica, B. holmesii, B. hinzii, B. pseudohinzii, B. ansorpii, B. trematum, B. avium, B. petrii, or any combination thereof.

In embodiments, the bacteria comprises an antibiotic resistant bacteria or a non-resistant bacteria.

In embodiments, the bacteria comprises a type 3 secretion system.

In embodiments, the infection comprises an acute infection or a chronic infection.

In embodiments, the infection comprises a respiratory infection or a non-respiratory infection.

In embodiments, the IL-1RA inhibitor comprises an anti-IL-1RA antibody. For example, the anti-IL-1RA antibody is a monoclonal antibody.

In embodiments, the IL-1RA inhibitor comprises a small molecule.

In embodiments, the method further comprises administering to the subject an antibiotic. In embodiments, the antibiotic comprises an antibacterial agent, an antifungal agent, and/or an antiviral agent.

In embodiments, the IL-1RA inhibitor is administered intranasally, intramuscular, orally or intraperitoneally.

Aspects of the invention are further drawn to a method of sensitizing an antibiotic resistant bacterial infection in a subject to an antibiotic. In embodiments, the method comprises administering to the subject an IL-1RA inhibitor.

In embodiments, the bacteria comprises Bordetella spp. For example, the bacteria comprises B. pertussis, B. parapertussis, B. bronchiseptica, B. holmesii, B. hinzii, B. pseudohinzii, B. ansorpii, B. trematum, B. avium, B. petrii, or any combination thereof.

In embodiments, the bacteria comprises a type 3 secretion system.

In embodiments, the infection comprises an acute infection or a chronic infection.

In embodiments, the infection comprises a respiratory infection or a non-respiratory infection.

In embodiments, the IL-1RA inhibitor comprises an anti-IL-1RA antibody. For example, the anti-IL-1RA antibody is a monoclonal antibody.

In embodiments, the IL-1RA inhibitor comprises a small molecule.

In embodiments, the method further comprises administering to the subject an antibiotic. For example, the antibiotic is an antibacterial agent, an antifungal agent, and/or an antiviral agent.

In embodiments, the IL-1RA inhibitor is administered intranasally, intramuscular, orally or intraperitoneally.

Other objects and advantages of this invention will become readily apparent from the ensuing description.

### BRIEF DESCRIPTION OF THE FIGURES

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1 shows eosinophils phagocytose and kill Bordetella bronchiseptica. (A) Eosinophils were challenged with RB50 (left) or RB50ΔbtrS (right) at an MOI of 10. At 4 hours post-infection samples were fixed and imaged in the transmission electron microscope. The image is representative of 4 independent experiments done in three technical replicates (n=4×3). (B) Intracellular bacteria were enumerated from at least 50-100 individual eosinophils. We did exclude death eosinophils. Bars represent in blue RB50 and red RB50ΔbtrS. The x-axis shows number of intracellular bacteria. (C) Eosinophils were challenged with RB50 (blue) or RB50ΔbtrS (red) at an MOI of 1. This experiment was performed in three individual biological replicates each of them containing 6 technical replicates. At different times post-infection samples were plated to enumerate bacteria and evaluate eosinophil killing. One-way ANOVA was performed ****p<0.0001

FIG. 2 shows Bordetella bronchiseptica blocks eosinophil pro-inflammatory responses via btrS mediated mechanism. Eosinophils were unchallenged (rhomboid black) or challenged with RB50 (circle blue) or RB50ΔbtrS (square red) at an MOI of 10. At 2- and 4-hours post-infection the supernatant was collected to performed a LegendPlex analysis. This experiment was performed in 4 individual biological replicates each one containing 3 technical replicates. Each symbol corresponds with the average of two technical replicates in the ELISA assay. (A) Bars shows pg/ml of IL9 measured at 2 hours post-infection. (B) Bars shows pg/ml of IL4 measured at 2 hours post-infection. (C) Bars shows pg/ml of IL2 measured at 2 hours post-infection. (D) Bars shows pg/ml of IL2 measured at 4 hours post-infection. (E) Bars shows pg/ml of IL17a measured at 2 hours post-infection. One-WAY ANOVA was performed to analyze the data. *p<0.05, **p<0.01, ***p<0.001, ns=non-significant. The values on (B) indicate the p-value

FIG. 3 shows Bordetella bronchiseptica promotes secretion of IL1RA by eosinophils via btrS mediated mechanism. Eosinophils were unchallenged (rhomboid black) or challenged with RB50 (circle blue) or RB50ΔbtrS (square red) at an MOI of 10. At 4 hours post-infection RNA was extracted to perform qRT-PCR. ΔΔct was normalized to actine. This experiment was performed in 3 individual biological replicates each one containing 3 technical replicates. Each symbol corresponds with the average of the three technical replicates in each qRT-PCR assay. (A) Bars shows mRNA levels (fold change) of IL1RA at 4 hours post-infection. (B) Bars shows mRNA levels (fold change) of IDO at 4 hours post-infection. (C) Bars shows mRNA levels (fold change) of PDL1 at 4 hours post-infection. One-WAY ANOVA was performed to analyze the data. ****p<0.0001, ns=non-significant.

FIG. 4 shows Bordetella bronchiseptica promotes secretion of IL1RA in vivo via btrS mediated mechanism. Balb/c mice were unchallenged (rhomboid black) or challenged with 30 μl of PBS containing 5×10⁵ RB50 (circle blue) or RB50ΔbtrS (square red). At different post-infection times lungs were collected to extract RNA and qRT-PCR was performed. DDct was normalized to actine. This experiment was performed in 2 individual experiments each one containing 3 mice of each group. Each symbol corresponds with the average of the three technical replicates in each qRT-PCR assay. (A) Lines and symbols show the average and SEM of mRNA levels (fold change) of IL1RA. (B) Lines and symbols show the average and SEM of mRNA levels (fold change) of IDO. (C) Lines and symbols show the average and SEM of mRNA levels (fold change) of PDL1. (D) At day 7 post-infection, lungs were collected in PBS and protease inhibitor. After homogenizing the supernatant was used to evaluate IL1RA ELISA. Each symbol represents the average of two technical replicates and the bars show the pg/ml of IL1RA secreted in lungs. One-WAY ANOVA was performed to analyze the data. *p<0.05; **p<0.01.

FIG. 5 shows IL1RA promotes Bordetella bronchiseptica persistence in the lungs. Balb/c mice were unchallenged (rhomboid black) or challenged with 30 μl of PBS containing 5×10⁵ RB50 (circle blue) or RB50ΔbtrS (square red) in two independent experiments containing between 2-4 mice each time. (A) At day 1, 3 or 5 post-infections, mice started treatment with anti-IL1RA antibodies until day 14 post-infection. Lung bacterial burden was enumerated. Symbols log 10 CFU of RB50. (B) At day 5 post-infection mice started daily treatment with intranasal anti-IL1RA. At day 14 bacterial burden was enumerated in the lungs. (C) At day 1 post-infection mice started daily treatment with intraperitoneal IL1RA. At day 14 post-infection bacterial burden was enumerated in the lungs. (D) Balb/c mice were intranasally infected with B. parapertussis. At day 5 we initiated daily treatments with anti-IL1RA up to day 14 post-infection (black). The other group was infected and untreated (green). At day 14 lung bacterial burden was enumerated. One-WAY ANOVA was performed to analyze the data, except figure (C and D) were T-test Wilcoxon was performed. *p<0.05; **p<0.01; ns=non-significant.

FIG. 6 shows bacterial T3SS promotes expression of IL1RA during Bordetella bronchiseptica infection. Balb/c mice were unchallenged (rhomboid black) or challenged with 30 μl of PBS containing 5×10⁵ RB50 (circle blue), RB50ΔbtrS (square red), or RB50ΔbscN (triangle green) in two independent experiments containing between 2-4 mice each time. (A) At day 7 post-infection, lungs were collected to extract RNA and perform qRT-PCR. ΔΔct was normalized to actine. This experiment was performed in 2 individual experiments each one containing 3 mice of each group. Each symbol corresponds with the average of the three technical replicates in each qRT-PCR assay. Bars represent mRNA levels (fold change) for IL1RA. (B) At day 7 post-infection lungs of 6 different animals per condition were fixed in 4% PFA followed by paraffin embedding, and sectioning. Immunofluorescence staining shows nucleus (blue, Hoechst), epithelial cells (red, Epcam), and IL1RA (green, anti-IL1RA). (C) Six Balb/c mice, three section of each were stained. Using the Keyence microscope, 10 areas of each section were acquired and the value of positive IL1RA staining per region of interest was evaluated using their software. Bars represent positive IL1RA signal per region of interest. One-WAY ANOVA was performed to analyze the data, except figure (C) were T-test Wilcoxon was performed. *p<0.05; **p<0.01; ***p<0.001, ****p<0.0001; and ns=non-significant.

FIG. 7 shows btrS blocks pro-inflammatory cytokine secretion from eosinophils. The secreted cytokine profile of bone marrow isolated eosinophils following infection with the wildtype B. bronchiseptica (blue) or RB50ΔbtrS (red) at an MOI of 10- and 4-hours post-infection. Unchallegened bone marrow isolated eosinophils (black). Supernatant was collected and used to perform a LegendPlex Thelper panel assay. IL17 (A), IL2 (B), and IL-1β (C) were increased in RB50ΔbtrS infected eosinophils. N=8(×2).

FIG. 8 shows btrS promotes IL-1RA expression by eosinophils. Bone marrow isolated eosinophils unchallenged (black) or challenged with the wildtype B. bronchiseptica strain (blue) or with the RB50ΔbtrS (red) for 4 hours at an MOI of 10−. At 4 hours RNA was extracted to determine fold-change of IDO (A), PD-L1 (B) and IL-1RA (C). N=7-12 (×3). Fold-change in IL-1RA, PD-L1, and IDO expression levels is shown. RNA was extracted with the PureLink kit from Invitrogen. qRT was performed using the one step Luna kit from NEB and in a BioRad thermocycler.

FIG. 9 shows btrS promotes IL-1RA expression by eosinophils. Lungs of Balb/c mice challenged with PBS (grey) or intranasally challenged with 30 μl of PBS containing 5×10^{\hat{ }}5 wildtype B. bronchiseptica strain (blue) or with the RB50ΔbtrS (red) for 4 hours. At 4 hours RNA was extracted to determine fold-change of IDA (A), PD-L1 (B) and IL-1RA (C). N=6(×3).

FIG. 10 shows btrS regulates an immunosuppressive pathway. A) C57BL/6J mice were intranasally inoculated with RB5O (blue) or RB50ΔbtrS (red) and CFUs were enumerated in the lungs at different time points. B) Balb/c (red) and GATA-1 (green) mice were intranasally inoculated with RB50ΔbtrS and euthanized at different times post-infection to enumerate colonies from the lungs at different time points.

FIG. 11 shows RB50ΔbtrS clearance requires eosinophils. Balb/c, GATA-1 C57BL/6J and EPX/MBP−/− mice were inoculated with RB50ΔbtrS (A) or RB50 (8). CFUs were enumerated from the lungs at day 14 post infection.

FIG. 12 shows btrS blocks eosinophil mediated killing. Bone marrow progenitors were differentiated into eosinophils and challenged with RB50 or RB50ΔbtrS. A) TEM of eosinophils challenged at MOI10 with RB50 at 4 hours-post-infection. B) Eosinophils were unchallenged (black) or challenged with RB50 (blue) or RB50ΔbtrS (red) at MOI 0.1 and CFUs were enumerated at different times post-infection. C) Eosinophils were unchallenged (black) or challenged with RB50 (blue) or RB50ΔbtrS (red) at MOI 10 and at 4 hours postinfection a LegendPlex was performed using the supernatant.

FIG. 13 shows RB5O promotes secretion of IL1RA by eosinophils. Eosinophils were challenged at MOI10 with media alone (black) or containing RB50 (blue) or RB50ΔbtrS (red). At 4 hours post-infection RNA was extracted to perform qRT-PCR of A) IL1RA, B) IDO, and C) PDL1.

FIG. 14 shows btrS regulates an immunosuppressive pathway. A) Balb/c mice were intranasally inoculated with PBS (black), RB5O (blue), or RB50ΔbtrS (red). At different times post-infection lungs were collected to perform qRT-PCR for IL-1RA. B) Balb/c mice were intranasally inoculated with PBS (black), RB5O (blue), or RB50ΔbtrS (red). At different times post-infection lungs were collected to perform IL-1RA Elisa. C) C57BL/6 or ilrn−/− mice were intranasally inoculated with RB5O (blue) or RB50ΔbtrS and euthanized at day 14 post-infection to enumerate colonies from the lungs. D) Balb/c mice were intranasally inoculated with B. parapertussis to be untreated (green) or treated (black) with anti-IL-1RA antibodies. Colonies were enumerated from the lungs at day 14 post-infection.

FIG. 15 shows bteA promotes IL1RA expression. Balb/c mice were intranasally inoculated with RB50 (blue) or different T3S5 mutants. At day 7 mice RNA from the lungs was extracted to perform qRTPCR for IL-1RA.

FIG. 16 shows IL-1RA increases at only 12 hours post infection. Balb/c mice were intranasally inoculated with RB5O or RB50ΔbtrS. At different times post-infection, lungs were fixed and section to perform fluorescence staining.

FIG. 17 shows model of early events during Bordetella spp. infection and long-term persistence.

FIG. 18 shows model that shows how bteA promotes IL1RA expression.

FIG. 19 shows that eosinophils mediate Bordetella clearance from the respiratory tract. (A) Acceleration of Bordetella clearance in an airway hyperresponsiveness model. C57BL/6 (wt and VIPR 2−/−) mice were infected intranasally with 30 μl of PBS containing ~5×10⁵ cfu of Bordetella strains RB50 and RB50ΔbtrS. Mice were sacrificed and CFU sampled at 7, 14, and 21 days post-infection. (B) Failure of bacterial clearance from the respiratory tract in eosinophil-deficient mice. Balb/c (red) or GATA-1−/− (green) mice were intranasally challenged with 50 μl of PBS containing 5×105 RB50ΔbtrS. Mice were euthanized and colonies were enumerated in the nasal cavity, trachea, or lungs. The graphs show the average and SEM. ****p<0.0001 using TWO-way ANOVA. N=22. (C) Eosinophils phagocytize B. bronchiseptica. Eosinophils were infected with RB50 at an MOI 10 for 4 hours, washed with PBS, fixed with paraformaldehyde, and imaged by electron microscopy.

FIG. 20 shows that btrS suppresses eosinophil effector function. (A) btrS suppresses eosinophil-mediated bacterial killing. Bone marrow-derived eosinophils from Balb/c mice were infected with Bordetella strains RB50 and RB50ΔbtrS at an MOI of 0.1, and CFU sampled at 30′, 1, 4, and 8 hours post-infection. Data represent mean and SEM of 3 independent experiments. ****p<0.00001. (B) btrS suppresses eosinophil histone activity. Differential proteomic analysis of lysates from eosinophil infected with RB50ΔbtrS for 4 hours and analyzed by label-free mass spectrometry. (C) Enhancement of eosinophil DNA trap formation in the absence of btrS. Eosinophils were infected with Bordetella for 4 hours at an MOI of 10 on coverslips coated with poly-D-lysine, fixed in 4% paraformaldehyde, stained for chondroitin sulfate, Bordetella LPS, and imaged by confocal microscopy.

FIG. 21 shows btrS promotes expression of IL-1RA and dampens chemokine and cytokine expression. (A) btrS is associated with higher IL1-RA expression in the mouse lung. Balb/c mice were infected with 30 μl of PBS containing approximately 5×108 cfu of B. bronchiseptica wildtype and ΔbtrS strains, and sacrificed at 7 dpi. IL1-RA expression was then quantified by qPCR in lung homogenates. Data represent mean and SEM of three biological replicates. btrS increased IL1-RA expression in eosinophils and induces alteration of cytokine in eosinophils. Quantification of IL1-RA (B), chemokines (C) and cytokines (D-E) in BM-derived eosinophils infected at an MOI of 0.1 are shown. Data represet means and SEM of 3 biological replicates. p=<0.0001****0.001***0.01**0.05.

FIG. 22 shows a working model indicating that the btrS sigma factor is associated with an increased expression of IL1-RA, which competively inhibits IL1-R1 receptor signalling, and subsequently suppresses eosinophil-mediated clearance of Bordetella.

### DETAILED DESCRIPTION OF THE INVENTION

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are drawn to new uses of IL-1RA inhibitors, such as treating a bacterial infection, preventing a bacterial infection, or sensitizing an antibiotic resistant bacterial infection in a subject to an antibiotic.

As referred to herein, the term “infection” can refer to a diseased state caused by an infectious agent of bacterial, viral, and/or fungal origin. For example, a bacterial infection can be the result of gram-positive bacteria, gram-negative bacteria or atypical bacteria. Accordingly, the term “bacterial infection” can refer to a condition in which a subject is infected with a bacterium. The infection can be symptomatic or asymptomatic.

Aspects of the invention are drawn towards methods of treating a bacterial infection in a subject. The term “treating” or “to treat” can refer to clinical intervention in an attempt to alter the natural course of the individual or subject being treated. For example, “treating a disease” can comprise partially or completely alleviating, ameliorating, improving, relieving, delaying onset of, inhibiting progression of, reducing severity of, and/or reducing incidence of one or more symptoms, features, or clinical manifestations of a bacterial infection. Clinical indications of treating or preventing a bacterial, viral, and/or fungal infection can comprise, for example, the complete or partial removal of signs or symptoms of the infection such as fever, headache, and/or isolation of bacterial, viral, or fungal colonies from samples. In embodiments, the treatment reduces or eliminates the infecting bacterial, viral, or fungal pathogen leading to microbial cure. In embodiments, the treatment does not reduce or eliminate the infecting pathogen, but alleviates the symptoms of the infection.

In embodiments, the treatment can be administered to a subject who does not exhibit signs of an infection, such as a bacterial infection (e.g., prior to an identifiable bacterial infection), and/or to a subject who exhibits only early signs of an infection, such as a bacterial infection, for the purpose of decreasing the risk of developing pathology associated with the bacterial infection.

As referred to herein, the term “bacteria” or its adjective “bacterial” can refer to members of a large domain of prokaryotic microorganisms. Ordinarily a few micrometers in length, bacteria have a number of shapes, ranging from spheres to rods and spirals and can be present as individual cells or present in linear chains or clusters of variable numbers and shape. In embodiments, the bacteria can be Bordetella spp., such as B. pertussis, B. parapertussis, B. bronchiseptica, B. holmesii, B. hinzii, B. pseudohinzii, B. ansorpii, B. trematum, B. avium, B. petrii, or any combination thereof.

Aspects of the invention are drawn towards methods of preventing a bacterial infection in a subject. The term “preventing” or “to prevent” can refer to the management and care of a subject for the purpose of hindering the development of the condition, disease or disorder, and can include the administration of the IL-1RA inhibitors to prevent or reduce the risk of the onset of symptoms or complications.

Aspects of the invention are also drawn towards methods of sensitizing an antibiotic resistant bacterial infection in a subject to an antibiotic. The term “sensitizing” or “to sensitize” can refer to raising the sensitivity or reducing the resistance of a subject to a therapeutic treatment, such as an antibiotic.

Aspects of the invention are also drawn towards methods of alleviating the symptoms of a bacterial infection in a subject. The term “alleviating a symptom of” can refer to relieving a symptom or making it less intense or severe.

In embodiments, the bacterial infection can be resistant to at least some types of antibiotics. An “antibiotic-resistant bacteria” can refer to a bacterial strain that has developed or is resistant to antibiotics due to genetic or non-genetic causes.

In embodiments, an IL-1RA inhibitor can be administered to a subject. The term “inhibitor” can refer to any agent which reduces the level and/or activity of a protein or protein complex. The term “inhibiting” can refer to decreasing, limiting, and/or blocking a certain action, function, or interaction. Non-limiting examples of inhibitors include small molecule inhibitors, degraders, antibodies, enzymes, or polynucleotides (e.g., siRNA). In some embodiments, a bacterial infection can be “inhibited” if at least one symptom of the infection is alleviated, terminated, slowed, or prevented. As used herein, an infection is also “inhibited” if recurrence of the infection is reduced, slowed, delayed, or prevented.

In embodiments, the IL-1RA inhibitor can be an antagonist. The term “antagonist” can refer to a compound or composition that can decrease, block, inhibit, abrogate, or interfere with a biological response by binding to or blocking a cellular constituent.

In embodiments, the IL-1RA inhibitor can be an antibody. The term “antibody” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples include a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the antigen and does not react with other polypeptides. In embodiments, the antibody can be an anti-IL-1RA antibody. In embodiments, the antibody can be a monoclonal antibody, such as an anti-IL-1RA monoclonal antibody.

In embodiments, the IL-1RA inhibitor can be a small molecule. The term “small molecule” can refer to a chemical compound that is small enough in size so that it can readily pass through a cellular membrane unassisted. In general, a small molecule can refer to a chemical compound that is not a polymer, such as a nucleic acid, polypeptide, or polysaccharide, although the term can encompass small polymers that can readily crossing the cellular membrane.

In embodiments, the IL-1RA inhibitor can be a peptide or peptidemimetic. The term “peptide” can refer to a macromolecule which comprises a multiplicity of amino or imino acids (or their equivalents) in peptide linkage. In the polypeptide or peptide notation used herein, the left-hand direction is the amino-terminal direction and the right-hand direction is the carboxy-terminal direction, in accordance with standard usage and convention. Peptides can include moieties other than amino acids (e.g., can be glycoproteins, proteoglycans, etc.) and/or can be otherwise processed or modified.

Peptides can contain L-amino acids, D-amino acids, or both and can contain any of a variety of amino acid modifications or analogs known in the art. Useful modifications include, e.g., terminal acetylation, amidation, glycosylation, biotinylation, substitution with D-amino acid or unnatural amino acid, and/or cyclization of the peptide. In some embodiments, peptides can comprise natural amino acids, non-natural amino acids, synthetic amino acids, and combinations thereof. A “short peptide” can refer to any peptide containing up to 25 amino acids (e.g., up to 20, 15, 12, 10, 9, 8, 7, 6, 5, 4, or 3). In some embodiments, a short peptide contains 5-25 amino acids. Peptides also can include peptidomimetics unless indicated otherwise. Herein the terminologies “mimic,” “mimetic,” “peptidomimetic” and the like can be used herein interchangeably.

In some embodiments, the IL-1RA inhibitor can be in a pharmaceutical composition comprising a pharmaceutically acceptable excipient, carrier, or diluent. In embodiments, a pharmaceutically acceptable excipient, carrier, or diluent can comprise any solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Pharmaceutical compositions can be in a form adapted to any route of administration, such as oral, subcutaneous, parenteral (intravenous, intraperitoneal, intradermal), intramuscular, rectal, epidural, intratracheal, inhalation, intranasal, transdermal (i.e., topical), transmucosal, vaginal, buccal, ocularly, or pulmonary administration, for example, in a form adapted for administration by a peripheral route, or is suitable for oral administration or suitable for parenteral administration. Other routes of administration are subcutaneous, intraperitoneal and intravenous, and such compositions can be prepared in a manner well-known to the person skilled in the art, e.g., as described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions can appear in conventional forms, for example, solutions and suspensions for injection, capsules and tablets, such as in the form of enteric formulations for oral administration. The composition can also be in a form suited for local or systemic injection or infusion and can, as such, be formulated with sterile water or an isotonic saline or glucose solution. The compositions can be in a form adapted for peripheral administration only, with the exception of centrally administrable forms. The compositions can be in a form adapted for central administration.

For example, pharmaceutical compositions suitable for injectable use can include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition can be sterile and can be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

In embodiments, sterile injectable solutions can be prepared by incorporating the IL-1RA inhibitor in the required amount in an appropriate solvent with one or a combination of ingredients enumerated herein, as required, followed by filtered sterilization. Dispersions are prepared by incorporating the IL-1RA inhibitor into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions can include an inert diluent or an edible carrier. They can be enclosed in gelatin capsules or compressed into tablets. For the purpose of oral therapeutic administration, the IL-1RA inhibitor can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral formula of the drug can be administered once a day, twice a day, three times a day, or four times a day, for example, depending on the half-life of the drug.

In embodiments, the terms “administration”, “administer”, or “administering” can refer to the physical introduction to the subject a composition containing the IL-1RA inhibitor using any of a variety of methods and delivery systems known to those of skill in the art. For example, the IL-1RA inhibitor can be administered intranasally, intramuscularly, orally, intravenously, pulmonary, subcutaneously, or intraperitoneally.

In embodiments, the IL-1RA inhibitor can be administered by bolus injection or by infusion. A bolus injection can refer to a route of administration in which a syrine is connected to the IV access device and the medication is injected directly into the subject. The term “infusion” can refer to an intravascular injection.

The IL-1RA inhibitor can be administered to a subject one time (e.g., as a single injection, bolus, or deposition). Alternatively, administration can be once or twice daily to a subject for a period of time, such as from about 2 weeks to about 28 days. It can also be administered once or twice daily to a subject for period of 1, 2, 3, 4, 5, 6, 7, 8, 9, 10, 11, 12 times per year, or a combination thereof.

In embodiments, the IL-1RA inhibitors can be administered to a subject in a therapeutically effective amount. A “therapeutically effective amount” or “therapeutically effective dose” can refer to that amount of the therapeutic agent sufficient to result in amelioration of symptoms, e.g., treatment, healing, prevention or amelioration of the bacterial infection, or an increase in rate of treatment, healing, prevention or amelioration of such conditions. When applied to an individual active ingredient administered alone, a therapeutically effective dose can refer to that ingredient alone. When applied to a combination, a therapeutically effective dose can refer to combined amounts of the active ingredients that result in the therapeutic effect, whether administered in combination, serially or simultaneously.

In embodiments, a therapeutically effective amount, such as a therapeutically effective amount of an IL-1RA inhibitor, can comprise a dose of less than about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject.

In embodiments, the therapeutically effective amount comprises less than about 0.1 mg, about 0.1 mg, about 0.5 mg, about 1.0 mg, about 2.5 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 70 mg, about 80 mg, about 90 mg, about 100 mg, about 120 mg, about 135 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 1.0 g, about 1.5 g, about 2.0 g, about 2.5 g, about 5 g, about 10 g, about 25 g, about 50 g, or more than 50 g.

A therapeutically effective amount of the IL-1RA inhibitor will depend on the age and weight of the subject and the concentration and/or formulation of the inhibitor.

In embodiments, the term “subject” or “patient” can refer to any organism to which aspects of the invention can be administered, e.g., for experimental, diagnostic, prophylactic, and/or therapeutic purposes. Typical subjects to which compounds described herein can be administered will be mammals, for example primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals, for example, pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted herein or another organism that is alive. The term “living subject” can refer to the entire subject or organism and not just a part excised (e.g., a liver or other organ) from the living subject. The terms “subject”, “individual”, and “patient” can be used interchangeably.

In embodiments, the bacterial infection can be caused by Bordetella spp. A Bordetella spp. infection can refer to an infection caused by one or more bacterial species of the genus Bordetella. Pathogens of the bacterial genus Bordetella cause respiratory disease in humans and animals. Non-limiting examples of such bacteria comprise B. pertussis, B. parapertussis, B. bronchiseptica. B. holmesii. B. hinzii, B. pseudohinzii, B. ansorpii. B. trematum. B. avium, B. petrii, or any combination thereof.

In embodiments, the bacteria can be an antibiotic resistant bacteria. As described elsewhere herein, an antibiotic resistant microorganism can survive exposure to an antibiotic.

In embodiments, the bacteria can be sensitive to antibiotics, and can thus be referred to as non-resistant bacteria. The term “sensitive” can refer to a microorganism such as bacteria wherein growth can be inhibited in the presence of a specific antibiotic.

In embodiments, the bacteria can comprise a type 3 secretion system (T3SS). One of the most widespread mechanisms that bacteria utilize to successfully block host immune responses is the type 3 secretion system. Many gram-negative bacteria utilize the T3SS to promote cytotoxicity, bacterial persistence, and decrease overall pro-inflammatory responses. Bordetella spp., for example, are among the gram-negative bacteria that harbor a functional T3SS.

In embodiments, the bacterial infection can be an acute infection or a chronic infection. An “acute infection” can refer to an infection characterized by a rapid onset of the disease, relatively short-term symptoms, and resolution within a few days. A “chronic infection” can refer to an infection that develops slowly and lasts a long time.

In embodiments, the infection can be a respiratory infection or a non-respiratory infection. A “respiratory infection” or “respiratory tract infection” can refer to an infection of a part of the body that is involved with breathing. A respiratory infection can be an infection of the upper respiratory tract (e.g., nose, ears, sinuses, and/or throat) or the lower respiratory tract (e.g., trachea, bronchial tubes, and/or lungs). Symptoms of an upper respiratory infection can include runny or stuffy nose, irritability, restlessness, poor appetite, decreased activity level, coughing, and fever. Symptoms of a lower respiratory infection can include cough, fever, chest pain, tachypnea, and sputum production. A “non-respiratory infection” can refer to an infection of a part of the body that is not involved with breathing.

Embodiments as described herein further comprise administering one or more additional active agents to a subject with the IL-1RA inhibitor. Non-limiting examples of such additional active agents can comprise a vaccine, an anti-inflammatory agent, a pain reliever, a steroid, or any combination thereof.

Embodiments can further comprise administering to the subject an antibiotic. The term “antibiotic” can refer to a substance that is antagonistic to the growth of microorganisms. Suitable antibiotics can be naturally-occurring, chemically-modified, or synthetically-produced. In embodiments, the antibiotic can be an antibacterial agent, an antifungal agent, and/or an antiviral agent. Non-limiting examples of antibiotics comprise penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

In embodiments, the IL-1RA inhibitor and the antibiotic can be administered sequentially or concurrently. “Sequential administration” can refer temporally separated administration of the IL-1RA inhibitor and the antibiotic. For example, the IL-1RA inhibitor and the antibiotic can be administered during a combination therapy for a time interval greater than about 15 minutes (about 20 minutes, 30 minutes, 40 minutes, 50 minutes, 60 minutes, or longer). “Concurrent administration” can refer to the simultaneous administration of the IL-1RA inhibitor and the antibiotic in any manner in which the pharmacological effects of the two agents are apparent in the patient. For simultaneous administration, the two agents need not be administered as a single pharmaceutical composition, in the same dosage form, or by the same route of administration.

### EQUIVALENTS

Those skilled in the art will recognize, or be able to ascertain, using no more than routine experimentation, numerous equivalents to the specific substances and procedures described herein. Such equivalents are considered to be within the scope of this invention.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—the Bordetella bronchiseptica Sigma Factor btrS Modulates Eosinophil-Mediated Bacterial Persistence Via the IL1-RA/IL-1β Axis

### Introduction

Bordetella is a genus of Gram-negative coccobacilli that mainly infects the respiratory tracts of mammals, and includes the human-adapted pathogen B. pertussis, the agent of whooping cough, a cause of substantial morbidity and mortality worldwide. Bordetella have evolved multiple mechanisms for subverting host immune responses to establish infection, encoding several virulence factors, such as toxins, which are regulated by multiple mechanisms including sigma factors, two component systems, or chaperones. One such example is the btrS sigma factor, which regulates immune modulatory pathways that suppress host responses to cause persistent infection.

Using a mutant lacking btrS, we established that eosinophils play critical roles during Bordetella bronchiseptica infection in the murine model. Mice lacking eosinophils presented a more persistent infection that resembles the infection caused by the wildtype bacteria, indicating that Bordetella spp. suppresses eosinophils functions to increase persistence. Disruption of eosinophil function by the B. bronchiseptica sigma factor via the IL1-RA/IL1-R1 axis can mediate bacterial persistence.

### Methods

In vivo experiments utilized normal, eosinophil-deficient and airway hyperresponsive mouse models. For in vitro assays, BM-derived eosinophils isolated from wildtype Balb/C mice were cultured and assays, including ELISA, qPCR, phagocytosis, microscopy, proteomics, were performed.

### Results

Eosinophils Mediate Bordetella Clearance from the Respiratory Tract

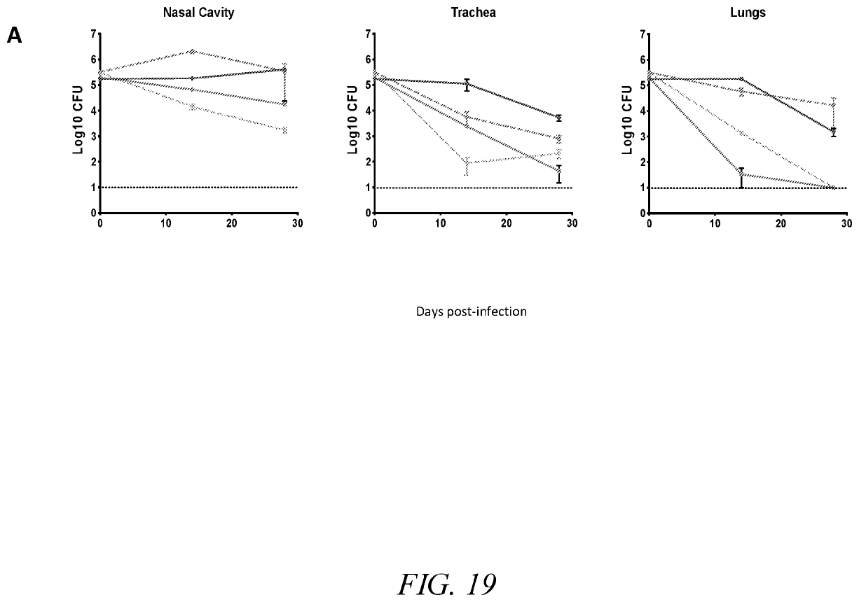

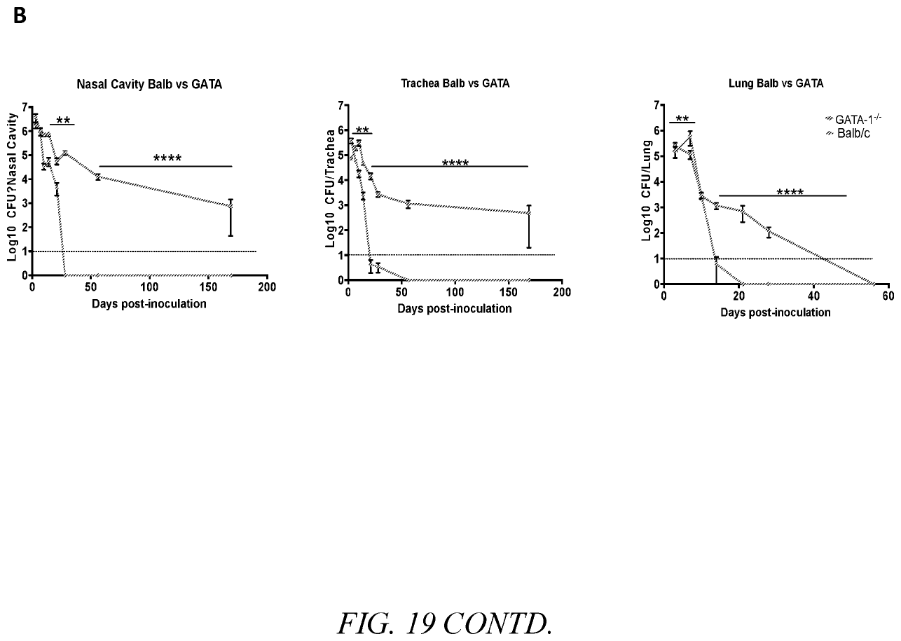

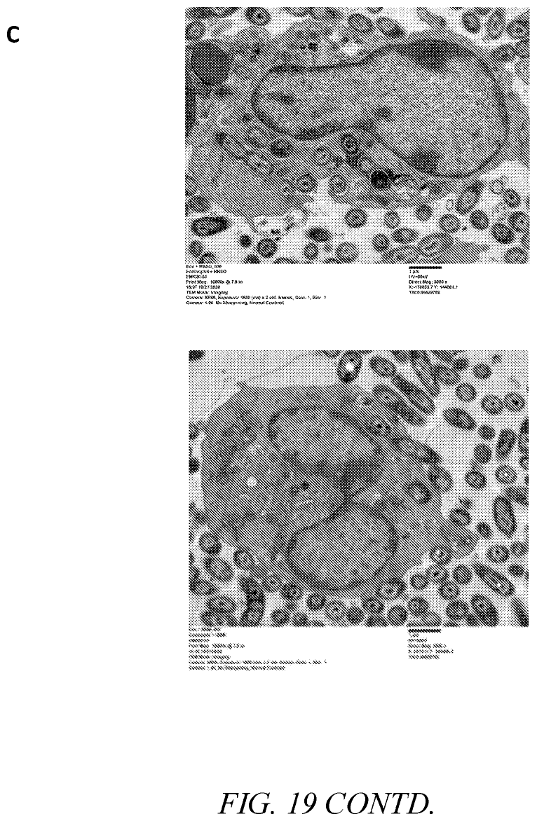

FIG. 19, panel A shows acceleration of Bordetella clearance in an airway hyperresponsiveness model. C57BL/6 (wt and VIPR 2^{-/-}) mice were infected intranasally with 30 μl of PBS containing ~5×10⁵ cfu of Bordetella strains RB50 and RB50ΔbtrS. Mice were sacrificed and CFU sampled at 7, 14, and 21 days post-infection. Panel B shows failure of bacterial clearance from the respiratory tract in eosinophil-deficient mice. Here, Balb/c (red) or GATA-1^{-/-} (green) mice were intranasally challenged with 50 μl of PBS containing 5×10⁵ RB50ΔbtrS. Mice were euthanized and colonies were enumerated in the nasal cavity, trachea, or lungs. The graphs show the average and SEM. ****p<0.0001 using TWO-way ANOVA. N=22. Panel C shows eosinophils phagocytize B. bronchiseptica. Here, eosinophils were infected with RB50 at an MOI 10, for 4 hours, washed with PBS, fixed with paraformaldehyde, and imaged by electron microscopy.

btrS Suppresses Eosinophil Effector Function

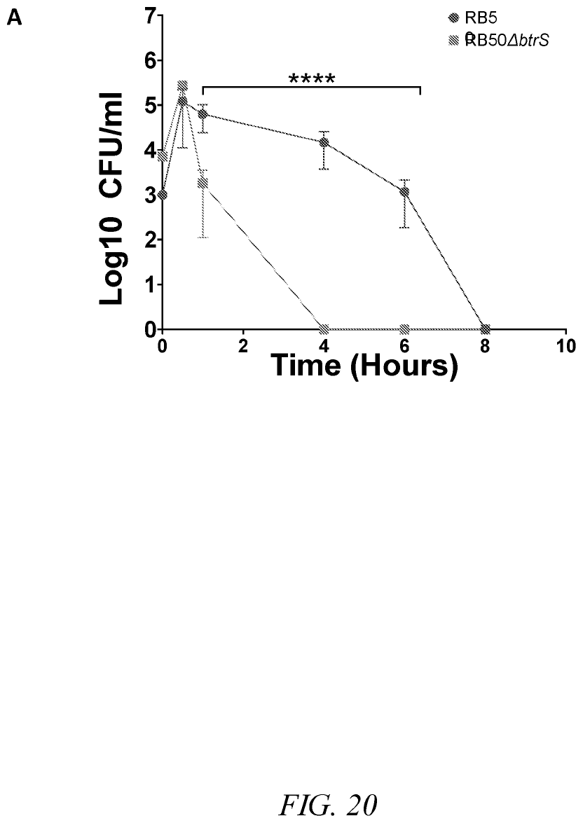

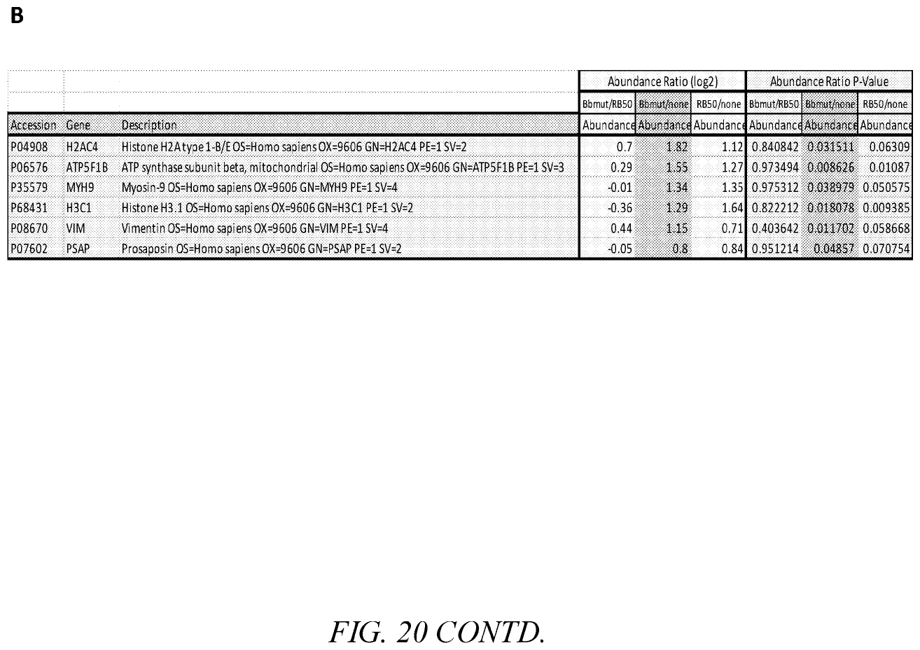

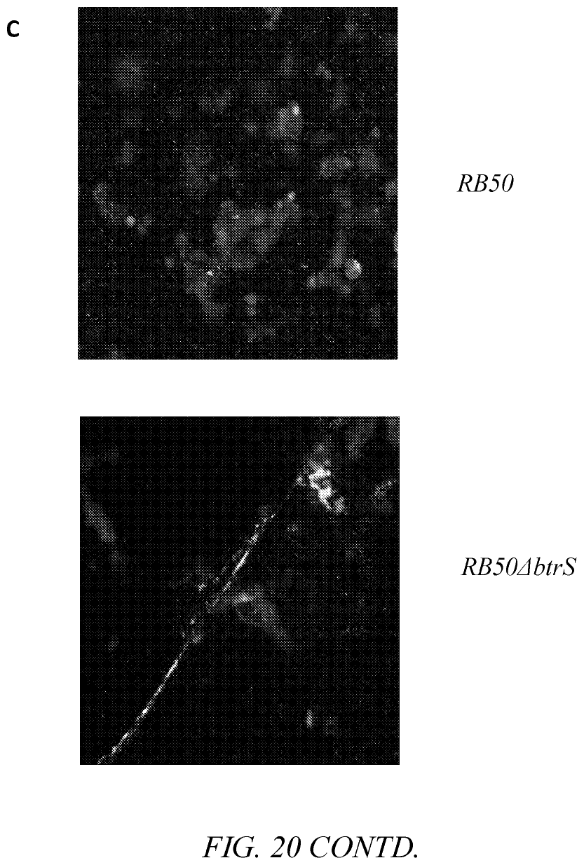

FIG. 20, panel A shows btrS suppresses eosinophil-mediated bacterial killing. Here, bone marrow-derived eosinophils from Balb/c mice were infected with Bordetella strains RB50 and RB50ΔbtrS at an MOI of 0.1, and CFU sampled at 30′, 1, 4, and 8 hours post-infection. Data represent mean and SEM of 3 independent experiments. ****p<0.00001. Panel B shows btrS suppresses eosinophil histone activity. Here, differential proteomic analysis of lysates from eosinophil infected with RB50ΔbtrS for 4 hours and analyzed by label-free mass spectrometry. Panel C shows enhancement of eosinophil DNA trap formation in the absence of btrS. Here, eosinophils were infected with Bordetella for 4 hours at an MOI of 10 on coverslips coated with poly-D-lysine, fixed in 4% paraformaldehyde, stained for chondroitin sulfate, Bordetella LPS, and imaged by confocal microscopy.

btrS Promotes Expression of IL-1RA and Dampens Chemokine and Cytokine Expression

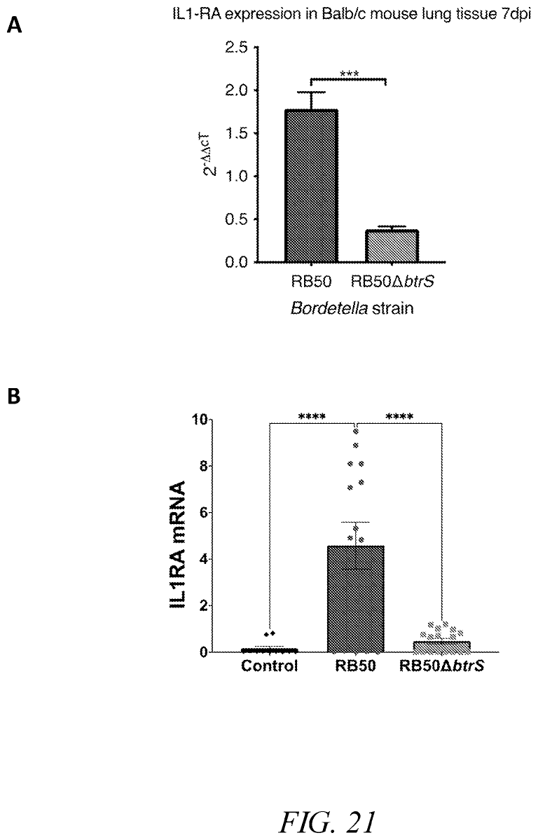

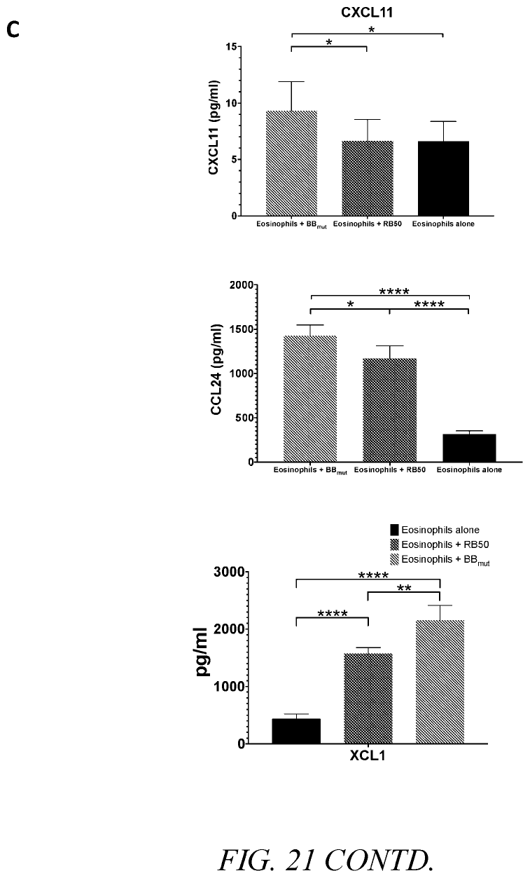

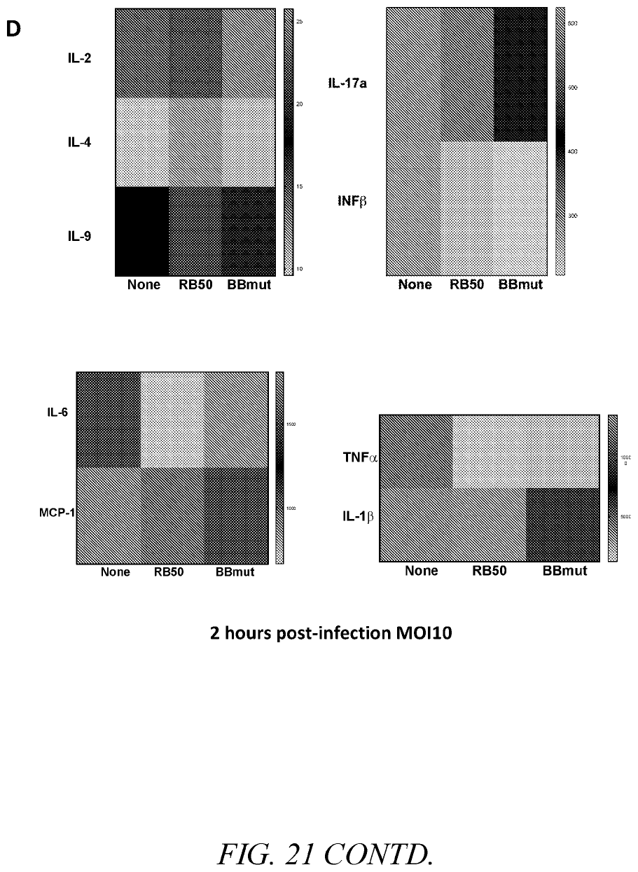

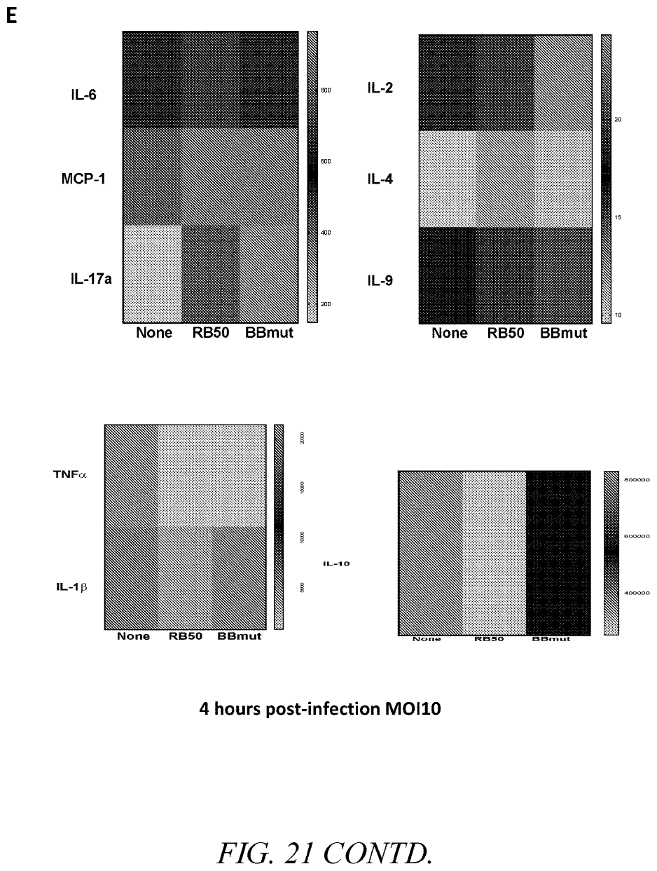

FIG. 21, panel A shows btrS is associated with higher IL1-RA expression in the mouse lung. Balb/c mice were infected with 30 μl of PBS containing approximately 5×10⁸ cfu of B. bronchiseptica wildtype and ΔbtrS strains, and sacrificed at 7 dpi. IL1-RA expression was then quantified by qPCR in lung homogenates. Data represent mean and SEM of three biological replicates, Panels B-D show btrS increased IL1-RA expression in eosinophils and induces alteration of cytokine in eosinophils. Quantification of IL1-RA (Panel B), chemokines (Panel C) and cytokines (Panel D-E) in BM-derived eosinophils infected at an MOI of 0.1 are shown. Data represet means and SEM of 3 biological replicates. p=<0.0001****0.001***0.01**0.05.

### Discussion

Our results demonstrate a btrS-associated increase in IL-1RA secretion by eosinophils mediating anti-inflammatory responses that alter immune function and increase bacterial persistence. This further provides evidence for a role of eosinophils in modulating host immunity during bacterial infection. Studies with other bacterial pathogens, such as H. pylori, also demonstrate induction of eosinophil-associated immunomodulatory molecules such as PDL-1, facilitating a cellular response that allows for bacterial persistence. This work, therefore, further provides the immunological and homeostatic functions of eosinophils in the context of a respiratory pathogen.

### Model

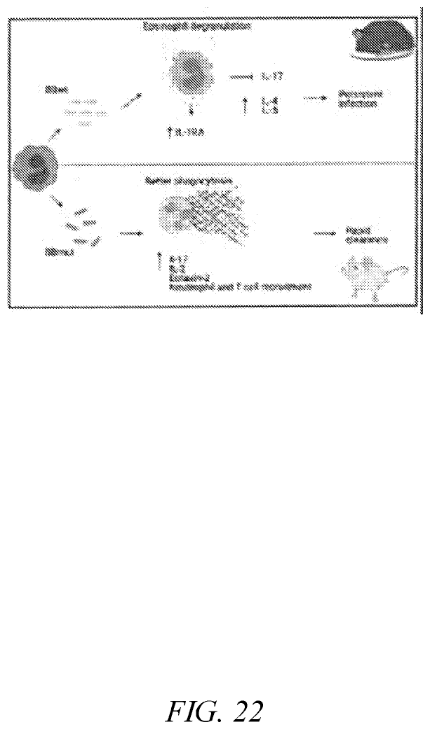

Our model, as shown in FIG. 22, provides that the btrS sigma factor is associated with an increased expression of IL1-RA, which competively inhibits IL1-R1 receptor signalling, and subsequently suppresses eosinophil-mediated clearance of Bordetella.

Experiments are ongoing to further identify the role of IL1-RA and other eosinophil-associated immunoregulatory molecules in suppression of the host response to Bordetella infection.

### REFERENCES CITED IN THIS EXAMPLE

1. Gestal M C, Rivera I, Howard L K, Dewan K K, Soumana I H, Dedloff M, Nicholson T L, Linz B, Harvill E T. Blood or serum exposure induce global transcriptional changes, altered antigenic profile, and increased cytotoxicity by classical bordetellae. Frontiers in microbiology. 2018 Sep. 7; 9:1969.
2. Gestal M C, Howard L K, Dewan K, Johnson H M, Barbier M, Bryant C, Soumana I H, Rivera I, Linz B, Blas-Machado U, Harvill E T. Enhancement of immune response against Bordetella spp. by disrupting immunomodulation. Scientific Reports. 2019 Dec. 30; 9(1):20261.
3. Gestal M C, Blas-Machado U, Johnson H M, Rubin L N, Dewan K K, Bryant C, Tiemeyer M, Harvill E T. Disrupting Bordetella immunosuppression reveals a role for eosinophils in coordinating the adaptive immune response in the respiratory tract. Microorganisms. 2020 Nov. 17; 8(11):1808.
4. Gestal M C, Whitesides L T, Harvill E T. Integrated signaling pathways mediate Bordetella immunomodulation, persistence, and transmission. Trends in microbiology. 2019 Feb. 1; 27(2):118-30.
5. Arnold I C, Artola-Borán M, Tallón de Lara P, Kyburz A, Taube C, Ottemann K, van den Broek M, Yousefi S, Simon H U, Müller A. Eosinophils suppress Th1 responses and restrict bacterially induced gastrointestinal inflammation. Journal of experimental medicine. 2018 Aug. 6; 215(8):2055-72.

### Example 2—Anti-IL-1RA as Treatment for Long-Term Bacterial Infections

One of the mechanisms that our body utilizes to dampen pro-inflammatory immune responses is by secreting a molecule known as IL-1RA, which is primarily produced by eosinophils and epithelial cells, although other immune cells can also secrete it. Anti-IL-1RA treatment has been used in auto-inflammatory diseases such as rheumatic disease, to halt the out-of-control pro-inflammatory responses that lead to tissue damage.

During my investigations that focus on understanding the role of eosinophils in promoting adaptive immune responses, I discovered that bacteria, such as Bordetella spp. can promote the secretion of IL-1RA to dampen host immune responses, decrease and delay the generation of adaptive immunity, and finally persist for long periods in the host, causing long-lasting persistent infections.

Without wishing to be bound by theory, an anti-IL-1RA antibody can be used for the treatment of bacterial infections. Non-limiting examples of such infections include those cause by Bordetella spp., such as respiratory infections or chronic lung infections, but also long-term choric infections caused by multi-drug resistant bacteria. This treatment will allow the immune system to control the infection allowing the generation of robust and prompt adaptive immune responses. As this is not targeted to kill bacteria, this can be a great alternative to be using for the therapy of antibiotic resistant bacteria, for which there is no current antibiotic treatment.

### Example 3

Infectious diseases are one of the greatest causes of morbidity, mortality and disability worldwide, and the current increase in antibiotic resistance is exponentially increasing the number of fatalities. Eosinophils are innate immune cells mostly known to be involved in asthma, allergies, viral infections, and immune responses to parasites. But eosinophils are also recognized to play a role in the immune responses to some bacterial pathogens. Eosinophils are key in maintaining homeostasis of the balance between Th1 and Th2 responses, mostly using 3 secreted molecules, IDO, PDL1 and IL1RA. Most of the recent literature that investigate the role of eosinophils during bacterial infections focus on the guts and have implicated these cells with the modulation of adaptive immune responses by two mechanisms, 1) by increasing expression of Program Death Ligand 1 that is used to trigger Th2 responses, and 2) via APRIL secretion promote the formation of MALT in the guts. In this context the findings reveal that Helicobacter pylori uses eosinophils to promote anti-inflammatory responses that enhance persistence. However, there is not clear evidence of the in-depth mechanisms by which eosinophils can promote pro-inflammatory responses or how bacteria manipulate eosinophils to enhance persistence.

Antibiotic resistance is one of the major threats of humankind and W.H.O. estimates a rate of 10 million deaths annually by 2050. Although, there is a current trend to revisit old remedies, such as old antibiotics; there is the imperative need to provide new therapeutical targets focus on improving host immune responses, rather than killing the bacterial threat. To determine the molecular mechanism that can be targeted against infectious diseases, we focus on understanding how bacteria manipulates host immune response with the expectation that unravelling the molecular mechanisms of host-pathogen interaction will provide us new drug and vaccine targets. We use Bordetella bronchiseptica and the murine model to identify the intricacies of the host-pathogen interaction.

Bordetella spp. are gram-negative coccobacilli that include 10 different species: B. pertussis, B. parapertussis, B. bronchiseptica, B. holmesii, B. hinzii, B. pseudohinzii, B. ansorpii, B. trematum, B. avium, and B. petrii. They are mostly respiratory pathogens characterized for producing a long-term persistent infection in humans and animals. B. pertussis and B. parapertussis are human pathogens and the etiological agents of whooping cough (pertussis), while B. bronchiseptica causes chronic infections in a broad range of animals, including immunocompromised humans. An important characteristic of B. bronchiseptica is that is a natural pathogen of mice on which causes a disease that mimics the pertussis in humans. Then combining the mice immunological tools with bacterial pathogenesis, we can validate the molecular mechanisms that underlie the host pathogen interaction. A recent increase in the number of cases of whooping cough has been detected, leading to CDC to recognized whooping cough as a reemerging infection disease of high priority. Only in 2014 over 24 million cases of whooping cough were diagnosed and still this illness is extremely underdiagnosed. The increase due to the lack of sterilizing protective immunity provided by the current acellular vaccine, possess a great risk not only for infants but also adults.

We have recently created a B. bronchiseptica mutant, herein BBmut or RB50ΔbtrS, that cannot suppress host immune responses, and is rapidly clear from the respiratory tract. Using the wildtype B. bronchiseptica and this mutant, BBmut, we can validate the immune cell populations and the immune signals that are critical for the development of a robust protective immunity. Our data demonstrate that eosinophils promote rapid clearance of Bordetella bronchiseptica infection from the mouse. A murine model lacking eosinophils revealed that mice without eosinophils fail to rapidly recruit B and T cells to the lungs and they had diminished cytokine secretion.

Our lab focuses on determining how eosinophils modulate adaptive immune responses to Bordetella spp. combining in vitro and in vivo approaches. Our new data demonstrates that bone marrow derived eosinophils challenged with BBmut, secrete proinflammatory cytokines and form eosinophils extracellular traps (also pro-inflammatory). Contrary, bone marrow derived eosinophils challenged with the wildtype strain of B. bronchiseptica, secrete Th2 type cytokines, block trap formation, and importantly, increase expression of IL-1RA. We determined whether in vivo mice challenged with the wildtype B. bronchiseptica strain can also increase the expression of IL-1RA, which is an anti-inflammatory molecule that halts pro-inflammatory immune responses. Our results revealed that infection with the wildtype strain of B. bronchiseptica promotes IL-1RA expression and secretion and that this can be one of the mechanisms that Bordetella spp, and other pathogens utilize to block efficient host-immune response and promote long term persistence. Accordingly, we will use anti-IL-1RA antibodies as an alternative treatment for infectious diseases.

### Example 4

Eosinophils are innate immune cells mostly known to be involved in asthma, allergies, viral infections, and immune responses to parasites. But eosinophils are also recognized to play a role in the immune response to some bacterial pathogens. Eosinophils are key in maintaining homeostasis of the balance between Th1 and Th2 responses, mostly using 3 secreted molecules, IDO, PDL1 and IL1RA. Some recent literature that investigates the role of eosinophils during bacterial infections focus on the guts and have implicated these cells with the dampening of adaptive immune responses by increasing expression of Program Death Ligand 1. Our previous work has revealed that Bordetella spp. harbor mechanisms to block eosinophils recruitment in the lungs and in fact the lack of eosinophils correlates with late clearance of infection. Similar to H. pylori, Bordetella spp. infections are characterized for being long-term persistent. Here, we combine in vitro and in vivo approaches to validate the role of eosinophils during adaptive response to Bordetella spp. infections.

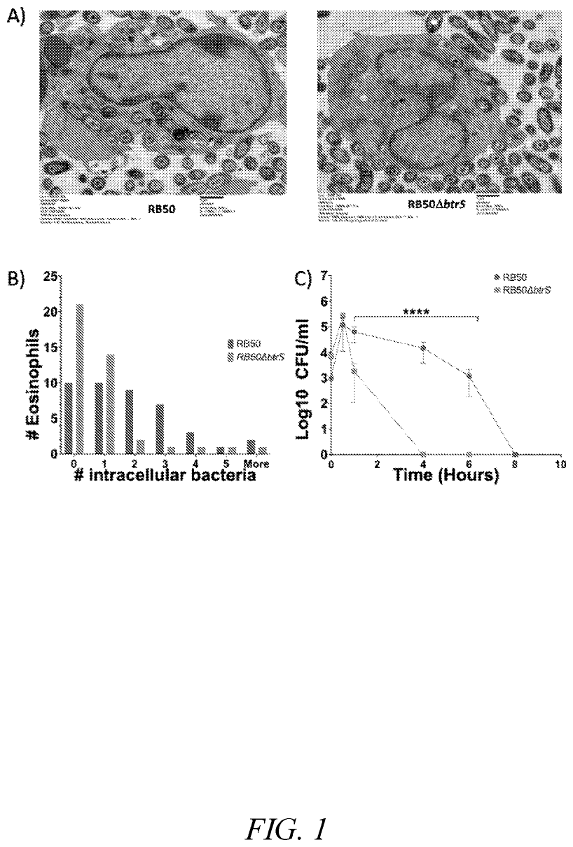

We first wanted to validate that eosinophils can phagocytose B. bronchiseptica wildtype (herein BBwt) and the mutant (herein BBmut). We differentiated eosinophils from bone marrow progenitors using previously stablished protocols (Mai et al 2019) and challenged them at an MOI of 10 with Bwt or BBmut. At 4 hours post-infection we fixed the samples and image them on the transmission electron microscope at our Pathology facility (FIG. 1).

Our results demonstrated that eosinophils can phagocytose both the wildtype and the mutant bacteria, and in fact, this happens via active phagocytosis. Previous literature indicates that eosinophils have bactericidal capabilities, so our next question was if eosinophils can kill the bacteria once it is phagocytosed. To answer that question, we followed the same experimental set up but instead fixing the samples, we plated them at different post infection times on Bordet-Gengou agar supplemented with 20 mg/ml of streptomycin and we enumerated colonies two days after incubation at 370° C. (FIG. 1 (C)).

Our results demonstrated that eosinophils have bactericidal capabilities, but interestingly, eosinophils kill BBmut in half of the time than they kill BBwt, indicating that the effector functions can be differentially activated following infection with BBwt or BBmut.

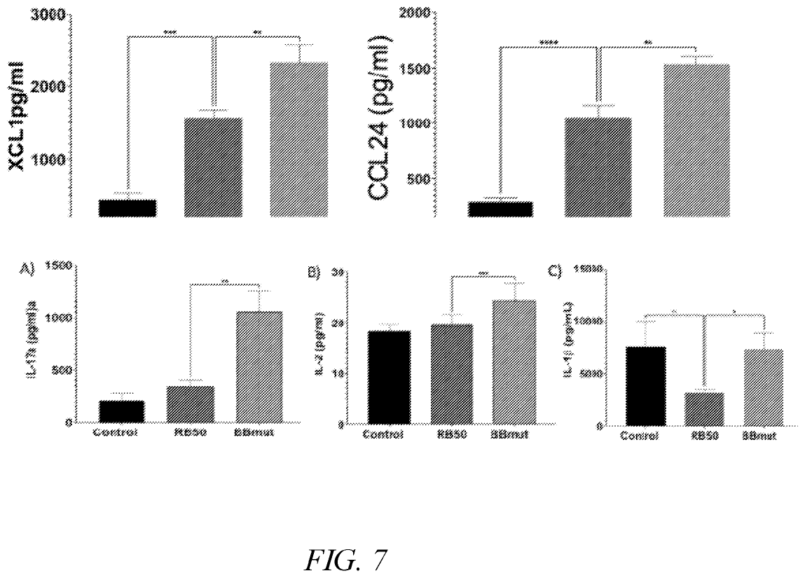

In order to determine the chemokine and cytokine profiling secreted by eosinophils after challenge with BBwt or BBmut, bone marrow derived eosinophils were challenged with BBwt or BBmut at an MOI of 10 and 4 hours post-infection the supernatant was collected to perform a LegendPlex assay (FIG. 7).

Our results show that following infection with BBmut, eosinophils secrete more lymphotactin as well as eotaxin-2. Interestingly, pro-inflammatory cytokines such as IL-17, IL-2 or IL-1b, were also more abundant in the supernatant of eosinophils that were challenged with BBmut. This indicates that RB50 is blocking the pro-inflammatory effector functions of the eosinophils and indicates an anti-inflammatory mechanism that is activated by infection with BBwt.

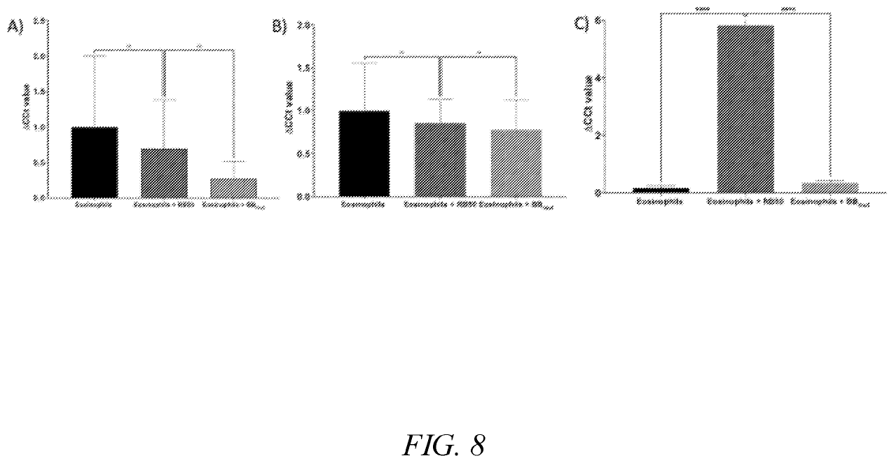

Eosinophils regulate the balance between pro- and anti-inflammatory responses mainly via three different molecules, 1) IDO (Indoleamine 2,3-dioxygenase), which suppresses T cells and Natural Killer cells, generate Tregs and myeloid-derived suppressor cells; 2) PD-L1 (Program death Ligand 1), which reduces the proliferation of antigen-specific T-cells in lymph nodes, reduces apoptosis in regulatory T cells, and promotes the apoptosis of Th1 T cells; and 3) IL-1RA (IL-1 Receptor Antagonist), which inhibits the IL-1 receptor signaling pathway blocking most of the pro-inflammatory responses. We wanted to determine whether infection with BBwt or BBmut activates anti-inflammatory signals in eosinophils. For that bone marrow derived eosinophils were challenged with BBwt or BBmut at an MOI of 10 and 4 hours post-infection RNA was extracted following PureLink manufacturer recommendations (FIG. 8).

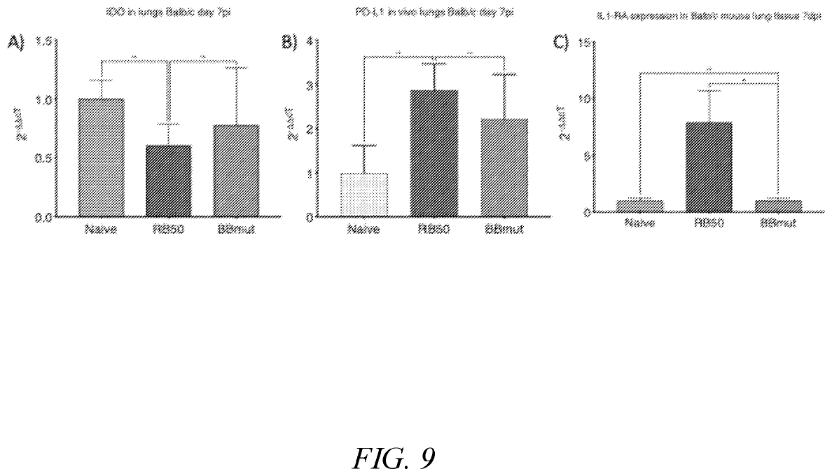

Our results demonstrate that while IDO and PD-L1 did not significantly changed the levels of expression, IL-1RA expression levels were significantly up-regulated following challenge with BBmut, indicating that BBwt harbor mechanisms that trigger anti-inflammatory responses mediated by IL-1RA. However, we wanted to further investigate if this pattern of expression was similar in vivo. For that we intranasally challenged mice with 30 mL of PBS alone or containing 5×10^{\hat{ }}5 BBwt or BBmut. At day 7 post-infection we collected the lungs to extract RNA and perform qRT-PCR (FIG. 9).

Our results demonstrate that again, levels of expression of IL-1RA are increased following infection with BBwt, indicating that this can be the mechanism that BBwt utilize in vivo in order to persist for long periods of time.

Without wishing to be bound by theory, anti-IL-1RA can be used as a therapeutic agent against long-term infections because the pro-inflammatory response won't be blocked, allowing for the generation of robust protective immunity.

### Example 5

Eosinophils are innate immune cells mostly known to be involved in asthma, allergies, viral infections, and immune responses to parasites. But eosinophils are also recognized to play a role in the immune response to some bacterial pathogens. Eosinophils are key in maintaining homeostasis of the balance between Th1 and Th2 responses, mostly using 3 secreted molecules, IDO, PDL1 and IL1RA. Most of the recent literature that investigate the role of eosinophils during bacterial infections focus on the guts and have implicated these cells with the modulation of adaptive immune responses by two mechanisms, 1) by increasing expression of Program Death Ligand 1 that is used to trigger Th2 responses, and 2) via APRIL secretion promote the formation of MALT in the guts. In this context the findings reveal that Helicobacter pylori uses eosinophils to pro-mote anti-inflammatory responses that enhance persistence. However, there is not clear evidence of the in-depth mechanisms by which eosinophils can promote pro-inflammatory responses or how bacteria manipulate eosinophils to enhance persistence. Previous reports indicate increased risk for allergy and asthma after vaccination or infection by several pathogens, including Bordetella spp. indicating a role for eosinophils during respiratory infections that is understudied.

Bordetella pertussis is rising, and outbreaks are being identified worldwide. In 2014 over 24 million cases of Bordetella spp. were diagnosed and probably this is an underestimation of the real burden. B. bronchiseptica (BB) is the evolutionary ancestor of B. pertussis, and they share more than 99% of their genetic content. Importantly, BB is a natural pathogen of mice that causes long-term persistence disease mimicking the characteristic chronic human pertussis infection. While determining the role of eosinophils during the generation of protective immunity against Bordetella spp., we have identified a new therapeutical target, anti-IL-1RA. Eosinophils challenged with the wildtype B. bronchiseptica strain secret anti-inflammatory molecules including IL-1RA, which function is to halt pro-inflammatory responses. By blocking IL-1RA secretion, we can promote a proper immune response that will lead to protective immunity.

Bordetella spp. are respiratory pathogens well known for being the causative agent of whooping cough. Currently, the cases of whooping cough are drastically increasing combined with also an emergence in the number of isolates that are antibiotic resistance, posing a risk to our infants as well as young adults who are the population that is suffering the most this resurgence. In our previous research we identified a Bordetella spp. regulator that controls the mechanisms by which Bordetella suppress host immune responses to colonize and promote long-term persistence. We have now identified the underlying host mechanism by which Bordetella spp. block immune responses. Infection with B. bronchiseptica promotes the expression of IL1RA, an anti-inflammatory molecule that suppresses host immune responses. Supplementation of IL1RA leads to increase persistence while using anti-IL1RA antibodies promotes rapid clearance of infection. Our work reveals that IL1RA can be used as an efficient treatment against Bordetella spp. infections and other multidrug resistant pathogens.

### Example 6—Bordetella Spp. Utilizes T3SS to Promote Secretion of IL1RA Leading to Long-Term Persistent Infection

### Abstract

A common feature of pathogens is their ability to suppress host immune responses. Understanding the molecular mechanisms and the common pathways that bacteria utilize to block host immune signaling cascade can provide new avenues for vaccine and therapeutic development. Preventable infectious diseases remain one of the major causes of morbidity and mortality worldwide and the current rise in antibiotic resistance is increasing this burden. Bordetella spp. are respiratory pathogens that cause the long-term illness known as whooping cough. Bordetella infections cause over 150,000 deaths each year, despite a vaccine being available. In our studies, we used the mouse pathogen B. bronchiseptica to investigate the pneumonic stage of disease, which very well mimics the fatal disease caused by B. pertussis. In our previous work we discovered a B. bronchiseptica mutant, RB50ΔbtrS, that clears rapidly from the lungs of mice and generates protective immunity that lasts for at least 15 months post-challenge. Combining the mouse immunological tools and both bacteria, the wildtype RB50 and mutant RB50ΔbtrS, which persists for up to 56 days and clears in 14-21 days, respectively, we investigated the mechanisms by which the wildtype B. bronchiseptica blocks host immune response to cause long term lung infection. Previous research indicated eosinophils as critical for rapid clearance of the mutant bacteria from the lungs. In vitro assays with eosinophils demonstrated that the RB50ΔbtrS mutant strain promotes the secretion of pro-inflammatory signals. In contrast, infection of eosinophils with RB50 promoted the secretion of anti-inflammatory signals such as IL1RA. Interestingly, IL1RA was also increased in the lungs of mice infected with the wildtype but not with the mutant RB50 strain. Infection with RB50ΔbscN, which lacks a functional type 3 secretion system (T3SS), was sufficient to prevent IL1RA induction indicating that the bacterial effector responsible for IL1RA upregulation is a substrate of the T3SS. Supplementation with IL1RA after infection with RB50 or RB50ΔbtrS resulted in increased lung bacterial burden for both bacterial strains. However, more rapid clearance of RB50 was observed after infection of mice in which IL1RA was knocked out. Furthermore, anti-IL1RA antibody treatment promoted rapid clearance of not only RB50 but also the human pathogens B. pertussis and B. parapertussis. Without wishing to be bound by theory, this indicates that IL1RA is a therapeutic target to treat severe cases of whooping cough.

Overall, this work demonstrates that Bordetella spp. induces IL1RA expression to promote persistence using the T3SS.

### Introduction

Infectious diseases are a major cause of morbidity and mortality worldwide¹. Due to the increase in antibiotic resistance and parental unwillingness to vaccinate children, the numbers of previously preventable deaths continue to rise¹. During evolution, pathogens evolved mechanisms to block host immune responses to prevent rapid clearance and allow for reinfection. Understanding the molecular mechanisms that bacteria utilize to suppress host immune responses and prevent the generation of protective immunity can provide new avenues for development of improved vaccines and therapeutics. In this work, we used the Bordetella bronchiseptica murine model to determine how these highly successful pathogens suppress host-immune responses. Here, we identify IL1RA as the mechanism by which Bordetella spp. promotes long-term persistence and this can be exploited in medical treatments against Bordetella spp, and possible other infections caused by bacterial pathogens that have a functional type 3 secretion system.

Bordetella spp. are respiratory pathogens also known for being the etiological agents of whooping cough. Classical Bordetella spp. comprises 3 different species; B. pertussis, B. parapertussis, and B. bronchiseptica, differing in their host range². B. bronchiseptica is the evolutionary ancestor of the other two species, sharing over 98% of the genetic content^{3,4}. Moreover, it is a natural pathogen of mice^{2,5} facilitating the study of host immunosuppression at the molecular level. Bordetella spp. infection has three main stages; the catarrhal stage, which is characterized by increased mucus secretion; the paroxysmal stage, during which constant violent episodes of cough cause apnea and even loss of consciousness; and the pneumonic phase, which is the main cause of fatal whooping cough⁶. Our work focuses on understanding the molecular mechanisms that Bordetella spp. utilize to suppress the generation of robust immune responses during the pneumonic fatal stage of disease. In our previous work, we focused on investigating how Bordetella spp. respond to inflammatory signals contained in blood and serum⁷. In response to blood and serum, we observed that Bordetella spp. upregulated expression of virulence factors as well as pathways that are known to help bacteria adapt to host microenvironments⁷. Importantly, amongst the different virulence factors, we also identified the increased expression of a sigma factor known as btrS. btrS is one of the many regulators of the T3SS⁸, however, it also regulates many other genes involved in virulence, metabolism, and other bacterial phenotypes^{9,10}. When studying the effects of btrS during the host-pathogen interaction, our results revealed that btrS regulates an immunosuppressive pathway that involves multiple known and unknown virulence factors. In the absence of btrS, there is a significant increase in the recruitment of B and T cells in the lungs at day 14 post-infection¹⁰. Mice infected with the btrS mutant develop long-lasting protective immunity that prevents reinfection with any of the three classical Bordetella spp. including the human pathogens B. pertussis and B. parapertussis, for at least 15 months post-vaccination¹¹. Excitingly, our results revealed that protective immunity was correlated with an increase in the numbers of eosinophils in the lungs¹².

Eosinophils are known for their function during parasitic infections as well as for their implications during allergic and asthmatic reactions¹³. The fact that eosinophils can kill bacteria has long been known¹⁴ however it has not gained significant attention until recently, when the role of eosinophil traps during infections, allergic reactions, and asthmatic processes was studied in-depth^{15,16}. Eosinophils are critical for immune homeostasis to keep the balance between Th1 and Th2 responses; in fact, they are pivotal to prevent mucosal autoinflammatory disorders¹³. Their anti-inflammatory function is performed through the secretion three main molecules; PDL1 (program death ligand-1), IDO (Indoleamine 2,3-dioxygenase), and IL1RA (IL1 receptor antagonist) 13. Eosinophils secrete one or more of these molecules to dampen proinflammatory responses and restore the immune homeostatic balance. During Helicobacter pylori infections, eosinophils' expression and secretion of PDL1 dampens pro-inflammatory responses allowing for persistent infections in the gastrointestinal tract¹⁷. However, this ability of eosinophils to modulate immune responses is not limited to the gut, as eosinophils are also found in other mucosal surfaces where they perform a critical role in maintaining homeostasis. Recent work from our lab demonstrated that during murine infection with a btrS Bordetella bronchiseptica mutant, RB50ΔbtrS, eosinophils are recruited to the lungs. In fact, they are critical for rapid clearance of the infection from the lungs and the generation of adaptive immune responses¹². Contrary, during infection with the wildtype RB50, eosinophils are not recruited to the lungs and infection persists for at least 56 days, indicating an immunosuppressive mechanism that can involve eosinophils.

Based on the literature on H. pylori¹⁷ and our previous results¹², we wanted to validate that wildtype Bordetella spp. promote anti-inflammatory responses that enhance colonization and persistence. Our results indicate that wildtype Bordetella spp. promote secretion of IL1RA by eosinophils and epithelial cells by promoting long term persistence. Supplementation with exogenous IL1RA during infection results in increased bacterial burden. Contrary to this, ablation of IL1RA leads to decreased lung colonization. Importantly, our results also revealed that anti-IL1RA can be used as treatment not only against B. bronchiseptica but also the human pathogen B. pertussis. Overall, our results provide a new insight into the role of eosinophils during infection as well as a new immunotherapy that can be used in multiple mucosal infections. We also demonstrated that bacteria promote secretion of IL1RA via T3SS, indicating that the use of anti-IL1RA therapies can be extended to numerous pathogens that use similar virulence factors.

### Results

### RB50 Suppress Eosinophil Mediated Killing

We have previously shown that Bordetella spp. can suppress host immune responses via btrS mediated mechanism¹⁰ in an eosinophil dependent manner¹², indicating that eosinophils can mediate adaptive mucosal immune responses. It has been shown that eosinophils can phagocytose and kill bacteria¹⁸⁻²⁰, and as a result they secrete cytokines that can promote pro- and anti-inflammatory responses^{13,21,22}. To validate eosinophils ability to phagocytose Bordetella spp., we differentiated bone marrow progenitors into mature eosinophils following previously published procedures²³. After confirming >98% differentiation with flow cytometry and/or cytospin, cells were challenged with RB50 or RB50ΔbtrS at a Multiplicity of Infection (MOI) of 10 for 4 hours to assess bacterial phagocytosis.

Using transmission electron microscopy, we observed that eosinophils can efficiently phagocytized both, RB50 and RB50ΔbtrS (FIG. 1, panel A). Interestingly, we observed that bacteria were being phagocytosed by the eosinophils using pseudopodia, in what was not a passive process but rather an active one. The electron microscope photos indicates that the number of bacteria internalized following infection with RB50 or RB50ΔbtrS, appear to be different. We counted the number of bacteria internalized by eosinophil (FIG. 1, panel B). Our results indicate that eosinophils challenged with RB50 contained in general between zero and three bacteria, although sometimes they can exceed more than 5 bacteria in the cytoplasm. Interestingly, eosinophils challenged with RB50ΔbtrS contained zero or one bacteria within the cytoplasm, indicating that RB50 intracellular survival in eosinophils was greater or that RB50ΔbtrS was more successfully phagocytosed and killed.

Next, we performed a killing assay. We inoculated eosinophils (98% differentiation rate) with an MOI of 1 of RB50 or RB50ΔbtrS and at different times post-challenge and bacterial colonies were enumerated (FIG. 1, panel C). Our results revealed that while RB50 is completely phagocytosed and killed by eosinophils in a period of at least 8 hours, RB50ΔbtrS is killed in half of the time (4 hours), indicating that the lower number of Rb50ΔbtrS can indicate that it is more efficiently phagocytosed and killed by eosinophils. Overall, our results indicate that both bacteria, RB50 and RB50ΔbtrS, can be phagocytosed by eosinophils. Moreover, the results also indicate that RB50ΔbtrS is killed more efficiently than the wildtype RB50.

### RB50 Suppress Eosinophil Mediated Pro-Inflammatory Responses

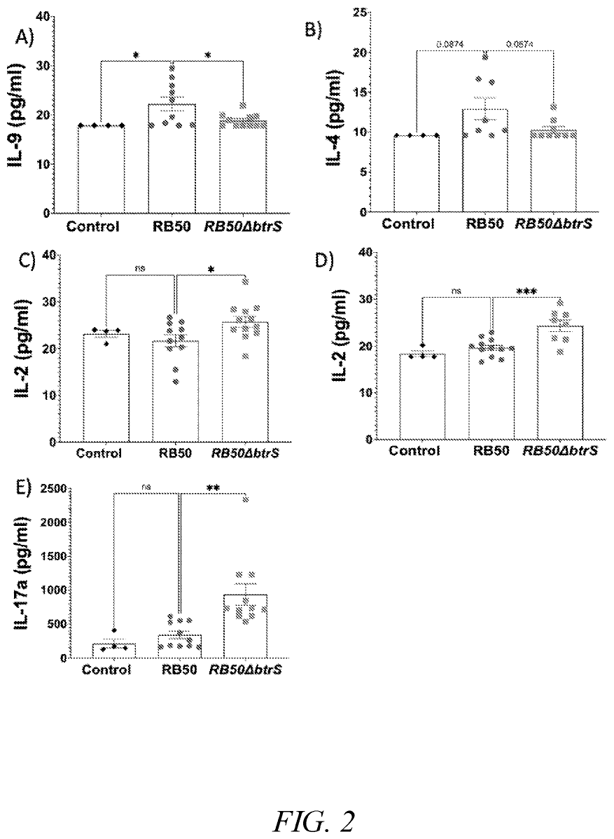

Eosinophils contain many cytokines and chemokines in their granules that can orchestrate host immune responses²⁴. Due to the differences observed on the interactions between eosinophils and wildtype RB50 or RB50ΔbtrS, cytokine signaling triggered by the RB50 or the RB50ΔbtrS mutant can be different. We infected eosinophils (98% differentiation rate) with RB50 or RB50ΔbtrS at an MOI of 10 and we investigated the cytokine secretome at 4 hours post-infection using the supernatant of our infected eosinophils. The results revealed that infection of eosinophils with RB50 promote an increase in the secretion of IL9 (FIG. 2, panel A) and IL4 (FIG. 2, panel B) at two hours post-infection compared with the uninfected control. However, eosinophils challenged with RB50ΔbtrS did not. Conversely, looking at pro-inflammatory cytokines, eosinophils challenged with RB50 did not increase the levels of secretion of IL2 and IL17 (FIG. 2, panels C-E). However, infection with RB50ΔbtrS significantly increased secretion of IL2 (FIG. 2, panel C) at only at 2 hours post-infection. At 4 hours post-infection, RB50ΔbtrS infected eosinophils presented an increased secretion of not only IL2 (FIG. 2, panel D) but also IL17 (FIG. 2, panel E). Overall, eosinophils challenged with RB50 wildtype B. bronchiseptica, secrete IL9 and IL4 which are cytokines associated with Th2 immune responses. Conversely, eosinophils challenged with RB50ΔbtrS secrete pro-inflammatory cytokines that promote Th1/Th17 responses.

### RB50 Promotes IL1RA Expression in Eosinophils

It is known that eosinophils prevent auto-inflammatory disorders on the mucosal surfaces, such as inflammatory bowel syndrome in the guts^{25,26} by secreting Interleukin-1 receptor antagonist (IL1RA)²⁷⁻²⁹, Program death ligand 1 (PD-L1)^{17,22}, and Indoleamine 2,3-dioxygenase (IDO)^{13,30}. Based on the differences identified in the cytokine profile, RB50 can promote the expression of one of these molecules to suppress the secretion of IL17 by eosinophils. Without wishing to be bound by theory, an increase in IL1RA will be responsible for suppressing IL17 secretion in eosinophils challenged with RB50²⁸.

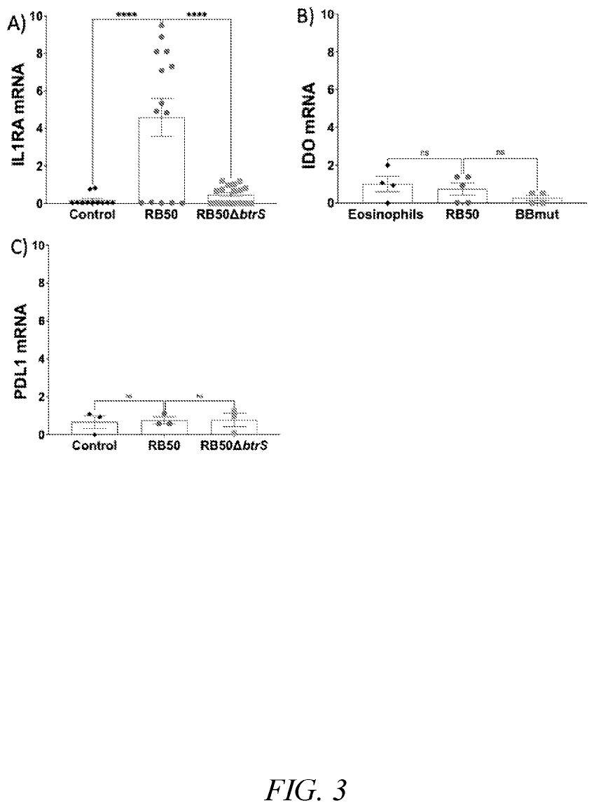

Using a minimalistic approach co-culturing bone marrow derived eosinophils (98% differentiation rate) challenged with both bacteria at an MOI of 10, we found that infection with RB50 but not RB50ΔbtrS promotes increase expression of mRNA levels of IL1RA by eosinophils (FIG. 3, panel A). No changes in the expression of IDO (FIG. 3, panel B) and PD-L1 (FIG. 3, panel C) were detected after infection with RB50 or RB50ΔbtrS compared with the uninfected control. Thus, these results indicate that RB50 promotes IL1RA expression in eosinophils, but not PD-L1 or IDO, indicating that RB50 promotes expression of IL1RA via a btrS mediated mechanism.

### RB50 Promotes Expression of IL1RA In Vivo

It has been previously shown that IL1RA has a role in vivo by aggravating infections³¹, one example being Staphylococcus aureus septicemia, where it has been shown that pathology is increased when mice are treated with IL1RA³². In addition, IL1RA has been shown to increase during infections with some bacteria^{31,33}. One example of IL1RA increasing is in cystic fibrosis patients infected with Pseudomonas aeruginosa³⁴.

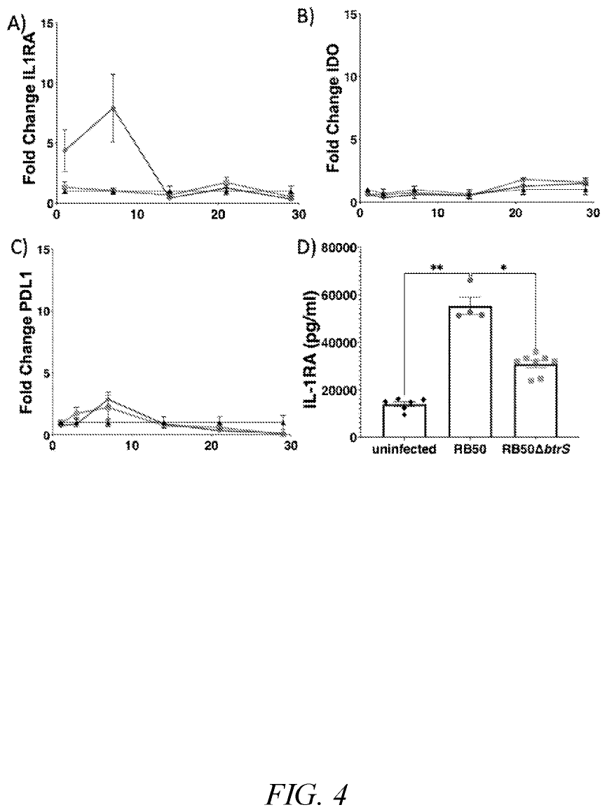

To evaluate the effects of RB50 and RB50ΔbtrS infection on IL1RA, PDL-1 and IDO in vivo, we challenged mice with PBS, RB50, or RB50ΔbtrS. At different times post-infection, we extracted RNA from the lungs to perform qRT-PCR and evaluate the changes in mRNA levels for IL1RA, IDO and PDL1 using actin to normalize the results. Following infection with RB50, the levels of IL1RA mRNA increase as early as day 1 post-infection, peaking at day 7 and then returning to basal levels at day 14 after infection (FIG. 4, panel A), showing a similar trend to that previously reported for P. aeruginosa infection³⁴. Interestingly the mRNA levels of IL1RA barely changed after infection with RB50ΔbtrS. Similar to our results in vitro, no changes on mRNA levels for IDO (FIG. 4, panel B) nor PDL1 (FIG. 4, panel C) were detected after infection with RB50.

To confirm that the changes in mRNA levels were related to differences in the protein levels, an ELISA using (with lung homogenized with protease inhibitors) was performed using lungs of infected mice at day 7 post-infection. This was the time that coincides not only with the peak of infection but also the peak in mRNA levels of IL1RA observed. Our results show that concentration of IL1RA in the lung homogenate increased at day 7 post-infection in mice challenged with RB50 (FIG. 4, panel D). Mice challenged with RB50ΔbtrS also presented a small increase but that was not comparable to that shown with the wildtype strain. Thus, our results revealed that infection with the wildtype bacteria RB50 promotes increased expression and secretion of IL1RA, an immunosuppressive molecule, that can enhance colonization and facilitate long term persistence.

### IL1RA Promotes Persistence In Vivo

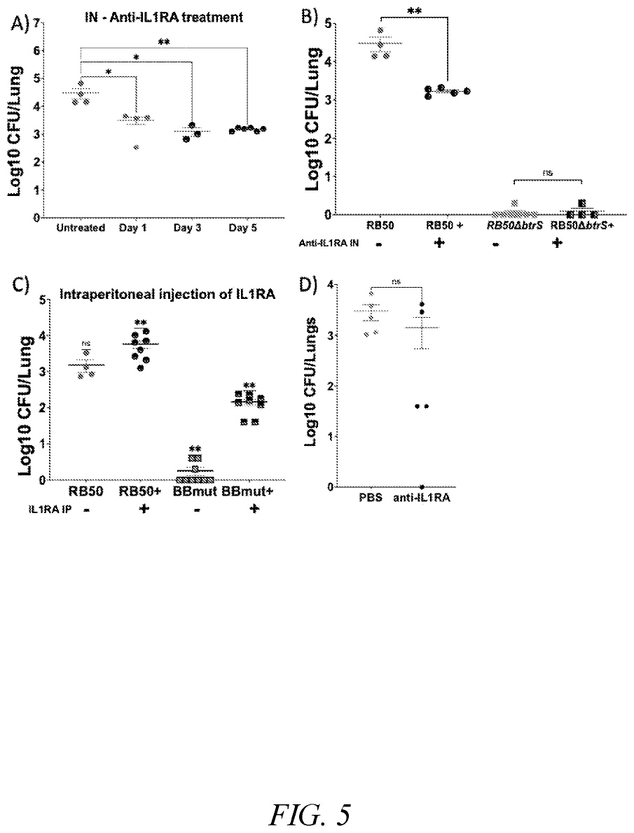

Previous literature associate supplementation with IL1RA in patients that receive treatment with anakinra and a worse outcome during infectious diseases, mostly correlated with increased bacterial burden^{32,35}. Due to the difference in persistence in lungs observed following infection with RB50 or RB50ΔbtrS¹⁰, we consider IL1RA can facilitate persistence. To determine the effects of IL1RA on length of infection with RB50 and RB50ΔbtrS, we used a knockout mouse model (Ilrln^{-/-})^{31,36} combined with the wildtype C57BL/6J. We evaluated lung colonization levels at day 14 post-infection with the RB50 and RB50ΔbtrS. We found that in the absence of IL1RA in the knockout mice bacterial burden of RB50 in the lungs was a decreased by 3 logs (FIG. 5, panel A). No differences were observed on mice infected with the RB50ΔbtrS mutant comparing Illrn^{-/-} and C57BL/6J mice. This was expected as by day 14, infection with RB50ΔbtrS is nearly cleared from the lungs.

To further confirm that the effects of IL1RA on bacterial persistence were due to the absence of IL1RA and not another reason, we used anti-IL1RA antibodies intranasally every 24 hours. We first evaluated when to start the treatment, so we infected mice with RB50 and started treatment at day 1, 3, and 5 post-infections (FIG. 5, panel A). We intranasally treated them daily until 14 and following euthanasia, we enumerated bacterial colonies in the lungs. We found that treatment with anti-IL1RA significantly decreased bacterial burden in the lungs. Interestingly, no differences were found in regards as to when to start the treatment. Starting treatment at day 1, 3 or 5 post-infections (FIG. 5, panel A) rendered the same reduction in the lungs bacterial burden. Based on these results we decided to start treatment at day 5 post-infection for the following experiments, as this day is the day at which whooping cough patients ordinarily report to the doctor.

We infected mice with RB50 and RB50ΔbtrS and provided treatment from day 5 up to day 14 and subsequently evaluated bacterial colonization in the lungs (FIG. 5, panel B). Our results reveal that treatment with anti-IL1RA antibodies significantly reduced bacterial burden following infection with RB50. However, similar to the results obtained with the IL1RA knockout mice, we did not observe decreased burden after infection with RB50ΔbtrS, because by day 14 the infection is already cleared.

We then wanted to validate that supplementation with IL1RA will promote increased bacterial persistence. We challenged mice with RB50 and RB50ΔbtrS. Twenty-four hours post-infection we began daily treatments with intraperitoneal injections of IL1RA until day 14 post-infection. Our results revealed that supplementation with IL1RA leads to an increase in bacterial burden during infection with RB50 and RB50ΔbtrS (FIG. 5, panel C), consistent with previous observations that IL1RA supplementation increases bacterial burden³⁷. Overall, these results indicate that IL1RA promotes bacterial persistence in the lungs. Contrary, ablation of IL1RA using a knockout mouse model or antibodies anti-IL1RA, results in more rapid clearance of B. bronchiseptica infections.

### Anti-IL1RA Antibodies Promote Rapid Clearance

Based on our previous results indicating that anti-IL1RA antibodies lead to decreased bacterial burden, we wanted to validate that anti-IL1RA antibodies can be used to promote clearance of infection by the three classical Bordetella spp. Groups of mice infected with the RB50 wildtype strain of B. bronchiseptica. While we did not reach statistical significance to more clearance of BP536 B. pertussis^{7,11}, and BPP12822 B. parapertussis^{7,11}, treated intranasally every day after day 5 post-infection with 10 ml of PBS or 10 ml of PBS containing 100 ng of anti-IL1RA antibodies. Our results clearly reveal a trend of less bacterial burden after treatment, and we will be testing different dosages and concentrations (FIG. 5, panel D). Overall, our results indicate that anti-IL1RA antibodies can be a treatment for Bordetella spp. infections.

### The Bacterial Type 3 Secretion System (T3SS) is Required for IL1RA Expression

Our data indicates that Bordetella spp. increase levels of IL1RA during infection to facilitate persistence. Moreover, treatment with anti-IL1RA antibodies promotes rapid clearance. To follow these findings, we investigated the bacterial mechanisms by which RB50 promotes IL1RA, while RB50ΔbtrS does not. btrSis a sigma factor that controls the expression of many known and unknown virulence factors including the type 3 secretion system (T3SS)⁸⁻¹⁰. T3SS is one of the most important bacterial virulence factors that many pathogens possess, known for promoting anti-inflammatory responses. In fact, during Bordetella spp. infections T3SS promotes an increase in IL10 after infection to facilitate colonization and persistence³⁸. Based on these data, T3SS can promote the expression and secretion of IL1RA as one of the multiple mechanisms by which the T3SS promotes anti-inflammatory responses.

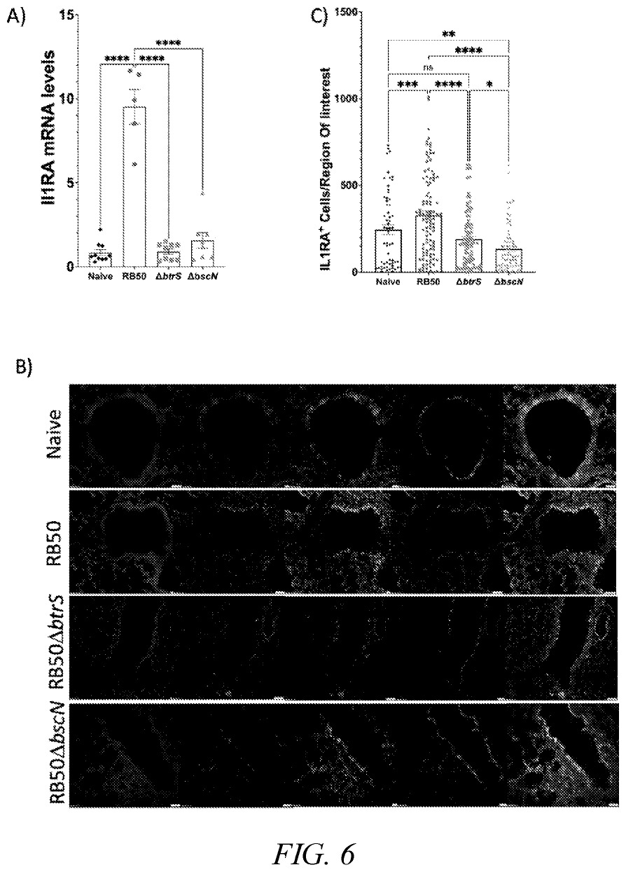

To validate this, we challenged mice with a T3SS mutant, RB50ΔbscN, which lacks the ATPase that allows the T3SS to pump effectors out of the bacteria cell³⁹. An important characteristic of this RB50DbscN mutant is that it clears more rapidly than the RB50⁴⁰ although not as fast as the RB50ΔbtrS, providing a good candidate to test our working model. We infected mice with RB50, RB50ΔbtrS, and RB50DbscN, at day 7 post-infection we evaluated levels of expression of IL1RA in the lungs of infected mice (FIG. 6, panel A). Indeed, mice infected with the RB50ΔbscN mutant did not present significantly increased levels of IL1RA in the lungs. RB50 revealed an increase in the levels of mRNA of IL1RA, while contrary, RB50ΔbtrS and RB50ΔbscN, did not increased expression of IL1RA compared to untreated controls, indicating that T3SS promotes IL1RA expression.

To further confirm the results, immunofluorescence microscopy was performed using paraffin embedded sections of the lungs of uninfected mice as well as day 7 post-challenged with RB50, RB50ΔbtrS, and RB50ΔbscN. We stained the nucleus (blue), IL1RA (green), and epithelial cells using Ep-Cam as a marker (red) (FIG. 6, panels B and C). Our naïve group have certain amount of constitutive IL1RA produced by several cells. These were expected as IL1RA is constitutively produced to maintain homeostasis and prevent auto-inflammatory reactions²⁸. The mice infected with the RB50 strain have a significant number of epithelial cells that were highly positive to IL1RA. Although eosinophils can contribute as important mediators of the Il1RA increase, epithelial cells are the major source. RB50ΔbtrS have lower IL1RA positive signal and it was localized only at the border of the epithelia. This was expected based on our mRNA, protein results and animal experiments. Mice challenged with RB50ΔbscN also have lower signal, similar to that obtained with our RB50ΔbtrS group, confirming the mRNA results previously obtained. Altogether indicating that the Bordetella T3SS promotes IL1RA as part of one of the multiple mechanisms by which T3SS dampen host-inflammatory responses.

### Discussion

Pathogens are characterized for their ability to suppress host immune responses². Most of the known mechanisms of bacterial pathogenesis focus on interactions between bacteria and classical phagocytes or innate immune cells, including macrophages⁴¹⁻⁴³ neutrophils^{44,45}, and dendritic cells^{46,47}. However, other immune cells classified as type 2, such as eosinophils’⁴⁸⁻⁵⁰ or mast cells⁵¹⁻⁵³, which have been historically overlooked during bacterial infections, are gaining attention. In this work we follow up on our previous findings to investigate the mechanism by which eosinophils mediate long term persistence of Bordetella spp. in the respiratory tract. Our results reveal that Bordetella spp. suppress eosinophil effector functions via btrS associated mechanism, promoting survival and eosinophil mediated killing. Moreover, eosinophils secrete IL1RA and this increase of IL1RA is also observable in lungs in vivo. Increased IL1RA promotes long term persistent infection in the lungs. Conversely, anti-IL1RA antibodies decrease lung bacterial burden after infection with any of the other classical Bordetella spp. Finally, we identify that the bacterial mechanism to promote IL1RA secretion is via T3SS, one of the most well-known bacterial virulence factors⁵⁴, indicating that this can be a conserved mechanism to promote immune-suppression.

Eosinophils are granulocytes mostly associated with pathological states of disease such as asthma or allergies^{16,48}. However, their role during immune homeostasis is known to be important especially in mucosal surfaces such as the gut, where eosinophils prevent autoinflammatory disorders such as Chron's or inflammatory bowel syndrome^{25,55}. Moreover, their function during cancerous processes is also being investigated as eosinophils play critical roles in the tumor microenvironment, where eosinophils are provided as a target for the development of future immunotherapies due to their crosstalk with lymphocytes and other immune cells⁵⁶. However, their role during infectious diseases has been overlooked with research focusing primarily on responses to parasitic infections^{57,58}. The fact that eosinophils can phagocytose and kill bacteria has been long known²⁰, and their ability to form traps during bacterial infections has been proven critical during infections with Staphylococcus aureus and other bacteria⁵⁹. In this work, we demonstrate that eosinophils can efficiently phagocytize and kill Bordetella spp. Importantly, we show that eosinophils can more effectively kill a mutant bacterium, RB50ΔbtrS which cannot suppress host immune response, than the RB50 wildtype, indicating that RB50 blocks eosinophil mediated killing via btrS mediated mechanism. Interestingly, when analyzing the differences in the cytokine profiling we identify that while eosinophils challenged with the RB50ΔbtrS mutant bacterium promote the secretion of pro-inflammatory cytokines, such as IL17 while on the contrary, eosinophils challenged with the wildtype increase the expression of anti-inflammatory cytokines such as IL1RA.

Recent literature demonstrates that eosinophils are utilized by bacteria to promote persistence in the gut¹⁷ and the respiratory tract¹². It has been shown that eosinophils in the gut dampen pro-inflammatory responses by secreting PDL1 in response to Helicobacter pylori infection facilitating persistence¹⁷. We have previously demonstrated that infection with RB50ΔbtrS promotes Th1/Th17 responses^{10,11}. Here our results indicate that in the respiratory tract, eosinophils, together with epithelial cells, secrete IL1RA in response to infection with B. bronchiseptica as an alternative mechanism to dampen pro-inflammatory responses.

IL1RA blocks IL1Receptor-1, competing with IL1β and IL1α for its binding and suppressing the subsequent immune signaling^{60,61}. IL1RA is used as a treatment for autoinflammatory disorders, and it is commercially available. Anakinra is used for the treatment of autoinflammatory diseases such as rheumatoid arthritis and others⁶². Patients who receive anakinra (IL1RA) during long periods of time, have higher risk of suffering from infectious diseases³⁷. But interestingly, while infections and bacterial burden increase, pathology can be decreased with IL1RA^{31,34,63}, highlighting the finely tuned balance that must be modulated during pro- and anti-inflammatory responses. Our results demonstrate that during infection with the wildtype bacteria which persist for up to 56 days in the lungs¹⁰, there is an increase in the levels of IL1RA at early stages of infection. Conversely, infection with our RB50ΔbtrS strain, which clears by day 14-21^{10,12}, does not present the same increase in IL1RA levels indicating that IL1RA can mediate the long-term persistence of the wildtype bacterial infection. Similarly, this increase in IL1RA has been previously shown following immunization with acellular pertussis⁶⁴, mRNA vaccines⁶⁵, and infections caused by Pseudomonas aeruginosa³⁴, or Chlamydia⁶⁶. Thus, indicating that pathogens can promote an increase of IL1RA to enhance colonization and persistence. We investigated the effects of anti-IL1RA antibodies during bacterial infection and our results revealed a decrease in the lung bacterial burden not only during infection with B. bronchiseptica, but also the human pathogens, B. pertussis and B. parapertussis.

Investigating the bacterial mechanism that drives the increase of IL1RA, our results indicate that it is mediated by the bacterial Type 3 Secretion System (T3SS). Previous research on the mechanisms of immune suppression of the T3SS focused on classical innate phagocytes⁵⁴, including macrophages⁶⁷, neutrophils⁴⁶, and dendritic cells⁶⁸. We identified a new mechanism of immunosuppression mediated by the T3SS that target eosinophils, and other immune cells, such as epithelial cells, to promote secretion of IL1RA. Overall, our findings demonstrate that bacteria utilize the T3SS to promote expression of IL1RA which then enhance colonization and long-term persistence. Treatment with anti-IL1RA antibodies decrease bacterial burden in the lungs, providing new avenues for therapeutic development. These results provide some insights into the side effects of long-term treatment with anakinra. Moreover, our results also highlight the need of a tight balance of the IL1 signaling axis which is required to prevent the auto-immune disorders, while allowing for the clearance of inflammatory diseases. Finally, IL1RA is also increased in fungal triggered asthma²⁹, which can further support that recurrent infections⁶⁹ can promote a chronic increase in IL1RA. This constant increase in IL1RA can reach a point from where it cannot be controlled anymore, leading to the development of asthma in infants.

### Materials and Methods

### Bacterial Strains and Culture Conditions

B. bronchiseptica strain RB50, a btrS knockout mutant¹⁰, and a bscN knockout mutant³⁸ were used in this study. B. bronchiseptica RB50 and mutants were cultured Difco Bordet-Gengou (BG) agar (BD, cat. 248200) and supplemented with 10% sheep defibrinated blood with 20 μg/mL streptomycin or classical LB broth as previously described⁷. Our B. pertussis strain BP536 and B. parapertussis BPP12822, were grow in BG agar or Stainer-Scholte as previously described⁷.

### Animal Experiments

Our animal experiments included Balb/c and il1rn^{-/-} mice. Our breeding colonies were kept under the care of the employees and veterinarians of Louisiana State University Health-Shreveport Animal Care Facility, Shreveport, LA Experiments were carried out in accordance with institutional guidelines.

For our animal inoculations mice were anesthetized with 5% isoflurane and when sleep they were intranasally challenged with 30-50 μl of PBS containing 1×10⁵ CFU/mL B. bronchiseptica RB50, RB50ΔbscN and RB50ΔbtrS.

Anti-IL1RA treatment was performed daily. Mice were anesthetized with 5% isoflurane and when asleep, they are intranasally inoculated with 10 μl of 500 mg of anti-IL1RA antibodies purchased from Leinco technologies (Anti Mouse/Human IL-1ra/IL-1F3-500 μg). We started the treatment at three different time points, day 1 post-infection, day 3 post-infection and day 5 post-infection.

Supplementation with IL1RA was performed daily from day 1 post-infection by intraperitoneally delivering 25 mg of murine IL1RA purchased from sigma (SRP6006-50UG). Mice were euthanized using 5% CO₂ followed by cervical dislocation. For animal protocols we followed the guidelines of AALAC, and the protocols described on our approved IACUC.

### Mouse Transcriptomics

At day 7 post-inoculation, mice were euthanized according with humane endpoints and our protocol approval. Lungs were collected in 4 different beads tubes containing Trizol and kept in ice for the shortest time possible before being homogenized and freeze at −20° C. For RNA extraction, we follow the protocols recommended by the manufacturer, PureLink RNA Extraction kit, Invitrogen, and the 4 different tubes were pull together to have the whole lung RNA contained in one only preparation.

Total RNA was extracted using the PureLink RNA Mini Kit (Invitrogen, Waltham, MA, United States) and treated with PureLink DNase (Invitrogen, Waltham, MA, United States) following manufacture protocols. RNA concentrations were quantified using a Nanodrop One (ThermoFisher Scientific, Waltham, MA, United States)⁷.

One μg of RNA was used for qRT PCR. To perform the qRT PCR we followed the recommendations of the manufacturer Luna Universal One-Step qRT-PCR Kit (New England BioLabs Inc., Ipswich MA, United States qRT PCR reactions were carried out in the Genomics Core Facility at Louisiana State University Health-Shreveport on a BIO-RAD CFX96 (LSU Health—Shreveport, LA, United States). Primer sequences are shown in table 1.

TABLE 1
Primer Sequences.

| | |
|---|---|
| IL-1RA Fw | AGT ACT GCC GAG GCC TGT AAT AA |
| IL-1RA RV | TTG TTC CTC AGG CCC CAA T |
| b-Actin Fw | TGG AAT CCT GTG GCA TCC ATG AAA C |
| b-Actin Rv | TAA AAC GCA GCT CAG TAA CAG TCC G |

### Immunohistochemistry

For pathology experiments, mice were euthanized and perfused intratracheally with sterile PBS and 4% paraformaldehyde (PFA). Lungs were subsequently fixed overnight in 10 mL of 4% PFA prior to processing and paraffin embedding. Lungs are processed, paraffin-embedded, sectioned in 0.5 μm thick slices, and placed on glass slides. Slides with PFA fixed and paraffin-embedded tissues were deparaffinized in consecutive washes in xylene, followed by rehydration in decreasing concentrations of ethanol ranging from 100%-50%. Staining was performed following previously published methods⁷⁰. Immunofluorescent images of the lungs were captured using a Keyence BZX-800 microscope. Image analysis was conducted using the Keyence Image Analysis Software.

### Statistical Analysis

Experiments were performed in three independent biological replicates. The exact number of mice and technical replicates is indicated in each figure legend. For animal experiments we performed Two-Way ANOVA analysis, while qRT-PCR experiments were analyzed using One-Way ANOVA analysis. Graphs and data were analyzed using the GraphPad V9.0. A p value≤0.05 was considered statistically significant. In the figures the asterisks correspond with *≤0.05, **≤0.01, ***≤0.001, and ****<0.0001.

### REFERENCES CITED IN THIS EXAMPLE

1. Baker R E, Mahmud A S, Miller I F, et al. Infectious disease in an era of global change. Nat Rev Microbiol. April 2022; 20(4):193-205. doi: 10.1038/s41579-021-00639-z
2. Gestal M C, Whitesides L T, Harvill E T. Integrated Signaling Pathways Mediate Bordetella Immunomodulation, Persistence, and Transmission. Trends Microbiol. February 2019; 27(2):118-130. doi: 10.1016/j.tim.2018.09.010
3 Parkhill J, Sebaihia M, Preston A, et al. Comparative analysis of the genome sequences of Bordetella pertussis, Bordetella parapertussis and Bordetella bronchiseptica. Nat Genet. September 2003; 35(1):32-40. doi: 10.1038/ng1227
4. Linz B, Ivanov Y V, Preston A, et al. Acquisition and loss of virulence-associated factors during genome evolution and speciation in three clades of Bordetella species. BMC Genomics. September 2016; 17(1):767. doi: 10.1186/s12864-016-3112-5
5. Bemis D A, Shek W R, Clifford C B. Bordetella bronchiseptica infection of rats and mice. Comp Med. February 2003; 53(1):11-20.
6. Kilgore P E, Salim A M, Zervos M J, Schmitt H J. Pertussis: Microbiology, Disease, Treatment, and Prevention. Clin Microbiol Rev. July 2016; 29(3):449-86. doi: 10.1128/CMR.00083-15
7. Gestal M C, Rivera I, Howard L K, et al. Blood or Serum Exposure Induce Global Transcriptional Changes, Altered Antigenic Profile, and Increased Cytotoxicity by Classical Bordetellae. Front Microbiol. 2018; 9:1969. doi: 10.3389/fmicb.2018.01969
8. Mattoo S, Yuk M H, Huang L L, Miller J F. Regulation of type III secretion in Bordetella. Mol Microbiol. May 2004; 52(4):1201-14. doi: 10.1111/j.1365-2958.2004.04053.x
9. Ahuja U, Shokeen B, Cheng N, et al. Differential regulation of type III secretion and virulence genes in Bordetella pertussis and Bordetella bronchiseptica by a secreted anti-σ factor. Proc Natl Acad Sci USA. March 2016; 113(9):2341-8. doi: 10.1073/pnas. 1600320113
10. Gestal M C, Howard L K, Dewan K, et al. Enhancement of immune response against Bordetella spp. by disrupting immunomodulation. Sci Rep. December 2019; 9(1):20261. doi: 10.1038/s41598-019-56652-z
11. Gestal M C, Howard L K, Dewan K K, Harvill E T. Bbvac: A Live Vaccine Candidate That Provides Long-Lasting Anamnestic and Th17-Mediated Immunity against the Three Classical. mSphere. Feb. 23, 2022; 7(1):e0089221. doi: 10.1128/msphere.00892-21
12. Gestal M C, Blas-Machado U, Johnson H M, et al. Disrupting. Microorganisms. November 2020; 8(11) doi: 10.3390/microorganisms8111808
13. Ondari E, Calvino-Sanles E, First N J, Gestal M C. Eosinophils and Bacteria, the Beginning of a Story. Int J Mol Sci. Jul. 27, 2021; 22(15) doi: 10.3390/ijms22158004
14. Baehner R L, Johnston R B. Metabolic and bactericidal activities of human eosinophils. Br J Haematol. March 1971; 20(3):277-85. doi: 10.1111/j.1365-2141.1971.tb07038.x
15. Simon D, Hoesli S, Roth N, Staedler S, Yousefi S, Simon H U. Eosinophil extracellular DNA traps in skin diseases. J Allergy Clin Immunol. January 2011; 127(1):194-9. doi: 10.1016/j.jaci.2010.11.002
16. Williams T L, Rada B, Tandon E, Gestal M C. “NETs and EETs, a Whole Web of Mess”. Microorganisms. December 2020; 8(12) doi: 10.3390/microorganisms8121925
17. Arnold I C, Artola-Borán M, Tallón de Lara P, et al. Eosinophils suppress Th1 responses and restrict bacterially induced gastrointestinal inflammation. J Exp Med. August 2018; 215(8):2055-2072. doi: 10.1084/jem.20172049
18. Persson T, Andersson P, Bodelsson M, Laurell M, Malm J, Egesten A. Bactericidal activity of human eosinophilic granulocytes against Escherichia coli. Infect Immun. June 2001; 69(6):3591-6. doi: 10.1128/IAI.69.6.3591-3596.2001
19. Yazdanbakhsh M, Eckmann C M, Bot A A, Roos D. Bactericidal action of eosinophils from normal human blood. Infect Immun. July 1986; 53(1):192-8. doi: 10.1128/IAI.53.1.192-198. 1986
20. DeChatelet L R, Migler R A, Shirley P S, Muss H B, Szejda P, Bass D A. Comparison of intracellular bactericidal activities of human neutrophils and eosinophils. Blood. September 1978; 52(3):609-17.
21. Woerly G, Roger N, Loiseau S, Capron M. Expression of Th1 and Th2 immunoregulatory cytokines by human eosinophils. Int Arch Allergy Immunol. 1999 February-April 1999; 118(2-4):95-7. doi: 10.1159/000024038
22. Gurtner A, Gonzalez-Perez I, Arnold I C. Intestinal eosinophils, homeostasis and response to bacterial intrusion. Semin Immunopathol. Jun. 6, 2021; 43(3):295-306. doi: 10.1007/s00281-021-00856-x
23. Mai E, Limkar A R, Percopo C M, Rosenberg H F. Generation of Mouse Eosinophils in Tissue Culture from Unselected Bone Marrow Progenitors. Methods Mol Biol. 2021; 2241:37-47. doi: 10.1007/978-1-0716-1095-4_4
24. Wechsler M E, Munitz A, Ackerman S J, et al. Eosinophils in Health and Disease: A State-of-the-Art Review. Mayo Clin Proc. October 2021; 96(10):2694-2707. doi: 10.1016/j.mayocp.2021.04.025
25. Jacobs I, Ceulemans M, Wauters L, et al. Role of Eosinophils in Intestinal Inflammation and Fibrosis in Inflammatory Bowel Disease: An Overlooked Villain? Front Immunol. 2021; 12:754413. doi: 10.3389/fimmu.2021.754413
26. Woodruff S A, Masterson J C, Fillon S, Robinson Z D, Furuta G T. Role of eosinophils in inflammatory bowel and gastrointestinal diseases. J Pediatr Gastroenterol Nutr. June 2011; 52(6):650-61. doi: 10.1097/MPG.0b013e3182128512
27. Yoshimura K, Ohge H, Uegami S, Watadani Y, Hirano T, Takahashi S. Small intestinal mucosa-associated lymphoid tissue lymphoma with deep ulcer and severe stenosis: A case report. Int J Surg Case Rep. November 2021; 88:106539. doi: 10.1016/j.ijscr.2021.106539
28. Sugawara R, Lee E J, Jang M S, et al. Small intestinal eosinophils regulate Th17 cells by producing IL-1 receptor antagonist. J Exp Med. Apr. 4, 2016; 213(4):555-67. doi: 10.1084/jem.20141388
29. Godwin M S, Reeder K M, Garth J M, et al. IL-1R A regulates immunopathogenesis during fungal-associated allergic airway inflammation. JCI Insight. Nov. 1, 2019; 4(21) doi: 10.1172/jci.insight.129055
30. McBrien C N, Menzies-Gow A. The Biology of Eosinophils and Their Role in Asthma. Front Med (Lausanne). 2017; 4:93. doi: 10.3389/fmed.2017.00093
31. Irikura V M, Hirsch E, Hirsh D. Effects of interleukin-1 receptor antagonist overexpression on infection by Listeria monocytogenes. Infect Immun. April 1999; 67(4):1901-9. doi: 10.1128/IAI.67.4.1901-1909.1999
32. Ali A, Na M, Svensson M N, et al. IL-1 Receptor Antagonist Treatment Aggravates Staphylococcal Septic Arthritis and Sepsis in Mice. PLoS One. 2015; 10(7):e0131645. doi: 10.1371/journal.pone.0131645
33. Giai C, Gonzalez C D, Sabbione F, et al. Staphylococcus aureus Induces Shedding of IL-1RII in Monocytes and Neutrophils. J Innate Immun. 2016; 8(3):284-98. doi: 10.1159/000443663
34. Iannitti R G, Napolioni V, Oikonomou V, et al. IL-1 receptor antagonist ameliorates inflammasome-dependent inflammation in murine and human cystic fibrosis. Nat Commun. Mar. 14, 2016; 7:10791. doi: 10.1038/ncomms10791
35. Zhao Y, Qin L, Zhang P, et al. Longitudinal COVID-19 profiling associates IL-1R A and IL-10 with disease severity and RANTES with mild disease. JCI Insight. Jul. 9, 2020; 5(13)doi: 10.1172/jci.insight.139834
36. Hirsch E, Irikura V M, Paul S M, Hirsh D. Functions of interleukin 1 receptor antagonist in gene knockout and overproducing mice. Proc Natl Acad Sci USA. Oct. 1, 1996; 93(20):11008-13. doi: 10.1073/pnas.93.20.11008
37. Salliot C, Dougados M, Gossec L. Risk of serious infections during rituximab, abatacept and anakinra treatments for rheumatoid arthritis: meta-analyses of randomised placebo-controlled trials. Ann Rheum Dis. January 2009; 68(1):25-32. doi: 10.1136/ard.2007.083188
38. Wolfe D N, Karanikas A T, Hester S E, Kennett M J, Harvill E T. IL-10 induction by Bordetella parapertussis limits a protective IFN-gamma response. J Immunol. February 2010; 184(3):1392-400. doi: 10.4049/jimmunol.0803045
39. Pilione M R, Harvill E T. The Bordetella bronchiseptica type III secretion system inhibits gamma interferon production that is required for efficient antibody-mediated bacterial clearance. Infect Immun. February 2006; 74(2):1043-9. doi: 10.1128/IAI.74.2.1043-1049.2006
40. Nicholson T L, Brockmeier S L, Loving C L, Register K B, Kehrli M E, Shore S M. The Bordetella bronchiseptica type III secretion system is required for persistence and disease severity but not transmission in swine. Infect Immun. March 2014; 82(3):1092-103. doi: 10.1128/IAI.01115-13
41. Ahmad J N, Sebo P. Adenylate Cyclase Toxin Tinkering With Monocyte-Macrophage Differentiation. Front Immunol. 2020; 11:2181. doi: 10.3389/fimmu.2020.02181
42. Kuwae A, Momose F, Nagamatsu K, Suyama Y, Abe A. BteA Secreted from the Bordetella bronchiseptica Type III Secetion System Induces Necrosis through an Actin Cytoskeleton Signaling Pathway and Inhibits Phagocytosis by Macrophages. PLoS One. 2016; 11(2):e0148387. doi: 10.1371/journal.pone.0148387
43. Lamberti Y A, Hayes J A, Perez Vidakovics M L, Harvill E T, Rodriguez M E. Intracellular trafficking of Bordetella pertussis in human macrophages. Infect Immun. March 2010; 78(3):907-13. doi: 10.1128/IAI.01031-09
44. Gorgojo J, Scharrig E, Gómez R M, Harvill E T, Rodríguez M E. Bordetella parapertussis Circumvents Neutrophil Extracellular Bactericidal Mechanisms. PLoS One. 2017; 12(1):e0169936. doi: 10.1371/journal.pone.0169936
45. Cerny O, Anderson K E, Stephens L R, Hawkins P T, Sebo P. cAMP Signaling of Adenylate Cyclase Toxin Blocks the Oxidative Burst of Neutrophils through Epac-Mediated Inhibition of Phospholipase C Activity. J Immunol. February 2017; 198(3):1285-1296. doi: 10.4049/jimmunol. 1601309
46. Fedele G, Schiavoni I, Adkins I, Klimova N, Sebo P. Invasion of Dendritic Cells, Macrophages and Neutrophils by the Bordetella Adenylate Cyclase Toxin: A Subversive Move to Fool Host Immunity. Toxins (Basel). September 2017; 9(10) doi: 10.3390/toxins9100293
47. Novák J, Fabrik I, Linhartová I, et al. Phosphoproteomics of cAMP signaling of Bordetella adenylate cyclase toxin in mouse dendritic cells. Sci Rep. November 2017; 7(1):16298. doi: 10.1038/s41598-017-14501-x
48. Jackson D J, Akuthota P, Roufosse F. Eosinophils and eosinophilic immune dysfunction in health and disease. Eur Respir Rev. Mar. 31, 2022; 31(163)doi: 10.1183/16000617.0150-2021
49. Dunn J L M, Rothenberg M E. 2021 Year in Review: Spotlight on Eosinophils. J Allergy Clin Immunol. Nov. 25, 2021; doi: 10.1016/j.jaci.2021.11.012
50. Jacobsen E A, Jackson D J, Heffler E, et al. Eosinophil Knockout Humans: Uncovering the Role of Eosinophils Through Eosinophil-Directed Biological Therapies. Annu Rev Immunol. Apr. 26, 2021; 39:719-757. doi: 10.1146/annurev-immunol-093019-125918
51. Johnzon C F, Rönnberg E, Pejler G. The Role of Mast Cells in Bacterial Infection. Am J Pathol. January 2016; 186(1):4-14. doi: 10.1016/j.ajpath.2015.06.024
52. Piliponsky A M, Acharya M, Shubin N J. Mast Cells in Viral, Bacterial, and Fungal Infection Immunity. Int J Mol Sci. Jun. 12, 2019; 20(12)doi: 10.3390/ijms20122851
53. Zimmermann C, Troeltzsch D, Giménez-Rivera V A, et al. Mast cells are critical for controlling the bacterial burden and the healing of infected wounds. Proc Natl Acad Sci USA. Oct. 8, 2019; 116(41):20500-20504. doi: 10.1073/pnas.1908816116
54. Kamanova J. Type III Secretion Injectosome and Effector Proteins. Front Cell Infect Microbiol. 2020; 10:466. doi: 10.3389/fcimb.2020.00466
55. Hogan S P, Rothenberg M E. Eosinophil Function in Eosinophil-associated Gastrointestinal Disorders. Curr Allergy Asthma Rep. February 2006; 6(1):65-71. doi: 10.1007/s11882-006-0013-8
56. Grisaru-Tal S, Rothenberg M E, Munitz A. Eosinophil-lymphocyte interactions in the tumor microenvironment and cancer immunotherapy. Nat Immunol. Aug. 24, 2022; doi: 10.1038/s41590-022-01291-2
57. Yoshimura K. [Mechanism of parasite killing by eosinophils in parasitic infections]. Nihon Rinsho. March 1993; 51(3):657-63.
58. Herndon F J, Kayes S G. Depletion of eosinophils by anti-IL-5 monoclonal antibody treatment of mice infected with Trichinella spiralis does not alter parasite burden or immunologic resistance to reinfection. J Immunol. December 1992; 149(11):3642-7.
59. Mukherjee M, Lacy P, Ueki S. Eosinophil Extracellular Traps and Inflammatory Pathologies-Untangling the Web! Front Immunol. 2018; 9:2763. doi: 10.3389/fimmu.2018.02763
60. Dinarello C A, van der Meer J W. Treating inflammation by blocking interleukin-1 in humans. Semin Immunol. Dec. 15, 2013; 25(6):469-84. doi: 10.1016/j.smim.2013.10.008
61. Dinarello C A, Simon A, van der Meer J W. Treating inflammation by blocking interleukin-1 in a broad spectrum of diseases. Nat Rev Drug Discov. August 2012; 11(8):633-52. doi: 10.1038/nrd3800
62. Mertens M, Singh J A. Anakinra for rheumatoid arthritis. Cochrane Database Syst Rev. Jan. 21, 2009; (1):CD005121. doi: 10.1002/14651858.CD005121.pub3
63. Chamekh M, Phalipon A, Quertainmont R, Salmon I, Sansonetti P, Allaoui A. Delivery of biologically active anti-inflammatory cytokines IL-10 and IL-1ra in vivo by the Shigella type III secretion apparatus. J Immunol. Mar. 15, 2008; 180(6):4292-8. doi: 10.4049/jimmunol. 180.6.4292
64. Armstrong M E, Loscher C E, Lynch M A, Mills K H. IL-1 beta-dependent neurological effects of the whole cell pertussis vaccine: a role for IL-1-associated signalling components in vaccine reactogenicity. J Neuroimmunol. March 2003; 136(1-2):25-33. doi: 10.1016/s0165-5728(02) 00468-x
65. Tahtinen S, Tong A J, Himmels P, et al. IL-1 and IL-1ra are key regulators of the inflammatory response to RNA vaccines. Nat Immunol. April 2022; 23(4):532-542. doi: 10.1038/s41590-022-01160-y
66. Rupp J, Kothe H, Mueller A, Maass M, Dalhoff K. Imbalanced secretion of IL-1beta and IL-1R A in Chlamydia pneumoniae-infected mononuclear cells from COPD patients. Eur Respir J. August 2003; 22(2):274-9. doi: 10.1183/09031936.03.00007303
67. Siciliano N A, Skinner J A, Yuk M H. Bordetella bronchiseptica modulates macrophage phenotype leading to the inhibition of CD4+ T cell proliferation and the initiation of a Th17 immune response. J Immunol. November 2006; 177(10):7131-8.
68. Skinner J A, Pilione M R, Shen H, Harvill E T, Yuk M H. Bordetella type III secretion modulates dendritic cell migration resulting in immunosuppression and bacterial persistence. J Immunol. October 2005; 175(7):4647-52.
69. Darveaux J I, Lemanske R F. Infection-related asthma. J Allergy Clin Immunol Pract. 2014 November-December 2014; 2(6):658-63. doi: 10.1016/j.jaip.2014.09.011
70. Tian X, Richard A, El-Saadi M W, et al. Dosage sensitivity intolerance of VIPR2 microduplication is disease causative to manifest schizophrenia-like phenotypes in a novel BAC transgenic mouse model. Mol Psychiatry. December 2019; 24(12):1884-1901. doi: 10.1038/s41380-019-0492-3

### Example 7

### A. Significance and Scientific Premise

Pathogens have been selected for their ability to suppress host immune response in order to colonize and cause long-term persistent infection. One of the most widespread mechanisms that bacteria utilize to successfully block host immune responses is the type 3 secretion system (T3SS)¹. Many gram-negative bacteria utilize the T3SS to promote cytotoxicity, bacterial persistence, and decrease overall pro-inflammatory responses². Bordetella spp. are among the gram-negative bacteria that harbor a functional T3SS. The T3SS in Bordetella spp. remains incompletely characterized, with only two effectors being identified thus far³: bteA, which induces high cytotoxicity to host cells⁴ by an undefined mechanism³, and bopN, which allows bteA translocation into the host cell⁵. In Bordetella spp, the T3SS is critical for long term-persistence^{6,7}, yet the molecular mechanisms by which the T3SS prevents rapid clearance are still not fully uncovered.

Bordetella spp., the causative agents of whooping cough-like disease, are highly transmissible respiratory pathogens (R₀=17)^{8,9} that encompass 3 species: B. pertussis (BP), B. parapertussis, and B. bronchiseptica (herein BB or RB50)¹⁰, with BB being the evolutionary ancestor of the other two¹¹. While BP causes disease exclusively in humans, BB can infect a wide variety of mammals, with infection of mice resembling a disease that is similar to the human pertussis, allowing for the study of the molecular mechanisms of bacterial pathogenesis in a natural setting of disease¹². Despite a vaccine being available the numbers of cases continue to rise¹³, Bordetella spp. has been registered as an emerging priority on the NIAID list of emerging infectious diseases/pathogens. In the search for new vaccines and therapies, we wanted to investigate how Bordetella spp. suppress host immune responses and we discovered the bacterial sigma factor, btrS¹⁴, which controls an immunosuppressive pathway¹⁵ that includes the T3SS¹⁴. Importantly, bacteria lacking btrS are more rapidly cleared from the lungs, leading to the generation of sterilizing immunity¹⁶.

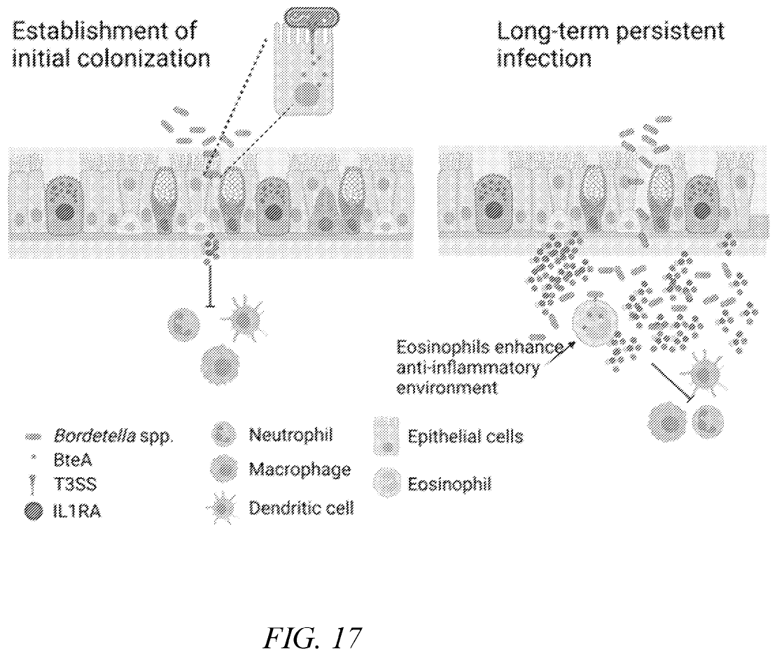

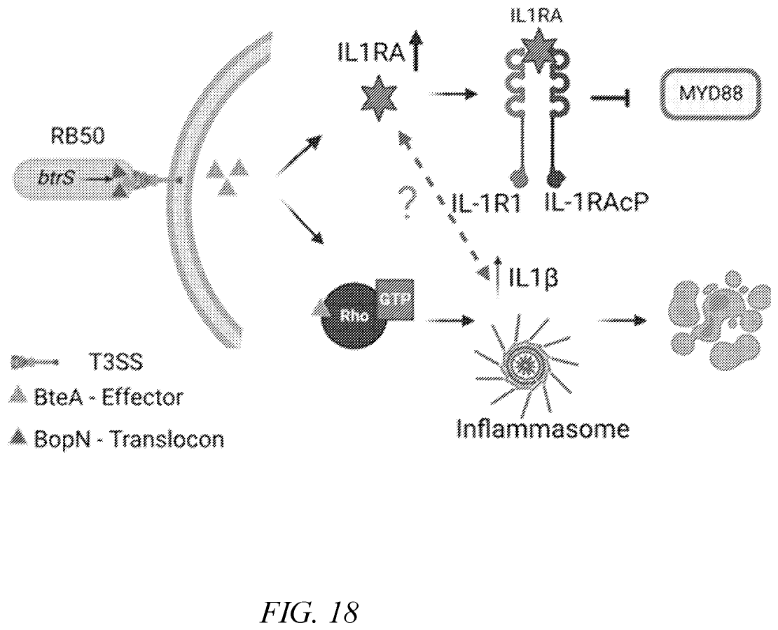

We observed that BB induces the expression of the anti-inflammatory cytokine Interleukin 1 receptor antagonist (IL1RA) in lungs of infected animals in a btrS-dependent manner. IL1RA induction is triggered by the T3SS and the T3SS effector, bteA. Our data showed that IL1RA secretion was essential to facilitate long-term persistence in the lungs, while ablation of IL1RA resulted in a more rapid decrease of bacterial burden. Based on our previous data, we generated a hypothetical model in which, early upon infection, Bordetella spp. promote expression of the T3SS via btrS, and injects the bteA effector into lung epithelial cells (first cells in contact with the bacteria) which leads to an increase in IL1RA induction. This increase in IL1RA will suppress immune cell recruitment allowing Bordetella spp. to establish infection and grow in numbers (FIG. 17). Then Bordetella spp. crosses the epithelial barrier, and other cells will also be injected with bteA to not only promote IL1RA induction, but also cell death. Thus, based on the similarities on functionality and sequence homology between the T3SS of Yersinia and Bordetella spp.³, bteA may interact with host kinases such as RhoGTPases to promote inflammasome activation and cell death (FIG. 18). Without wishing to be bound by theory, bteA acts at both stages by directly increasing IL1RA and indirectly to promote host inflammasome activation, which leads to increase in IL1γ and subsequently IL1RA. This combined facilitated early events of colonization and bacterial growth, allowing bacteria to cross the lung epithelium and reach deeper tissue (FIG. 17). To our knowledge, this is a new area of investigation in the Bordetella spp. field focusing on bacterial mechanisms that facilitate early events of colonization mediated by the T3SS effector bteA, and how the bteA also contributes to the long-term persistent lung infection by activating inflammasome and subsequent cell death.

IL1RA is currently used as a therapy during autoinflammatory diseases and cancer therapies because of its critical role of IL1 regulation in cancer progression. This indicates its potential use during other diseases such as infection. Significantly, our findings will define at least one mechanism by which the T3SS effector, bteA. promotes anti-inflammatory responses in the host and leads to early events of colonization and long-term persistence. Our results will help us to understand the first steps that are critical for the establishment of and subsequent disease, providing a new understanding of early events that can lead to new therapeutical targets. Without wishing to be bound by theory, our preliminary data indicate that IL1RA is also responsible for long-term persistence in the lungs. In fact, anti-IL1RA therapy appears to be a promising candidate for the treatment of Bordetella spp. lung infections. We recently demonstrated that eosinophils play an important role during Bordetella spp. infections¹⁷ and clearance of btrS-deficient bacteria. These findings have opened new scientific questions and led to other investigations focused on the role of eosinophils during Bordetella spp. transmission¹⁸ and therapeutical approaches against asthmatic and allergic processes¹⁹.

These published and preliminary findings led us to hypothesize that Bordetella spp. promote IL1RA expression in a bteA-T3SS dependent manner to facilitate early events of colonization/infection and increased bacterial persistence in the lungs. This proposal will broaden our understanding of the molecular mechanisms by which the T3SS promote the establishment of the infection, promotes IL1RA to also increase persistence, and how interferes with the host cell signaling. We will gain knowledge on the mechanism of action of the bteA effector in Bordetella spp. Moreover, as we are including BB and BP which differ in only one aminoacidic (Ala503 insertion). This will allow us to learn about the importance of this insertion during evolution in regard to the gain/loss of functionality⁴ and host-immune suppression. The finding that bacteria utilize T3SS to promote IL1RA provides a better understanding of immunosuppressive mechanisms. Also, our results point at a new role for epithelial cells as primary target of Bordetella, and other pathogens, to establish infection. Because btrS and T3SS are conserved genes in other bacterial species, we predict the knowledge gained can apply to other respiratory bacteria that use similar mechanisms to subvert host immunity, meaning our observations have the potential to be the catalyst of a paradigm shift in the way respiratory infections are treated.

### B. Innovation

Here, we investigate the mechanism by which Bordetella spp. utilize the T3SS effector, bteA, to induce IL1RA and facilitate early events of colonization/infection and promote long-term persistence. The attribution of a new role for the BB/BP T3SS in promoting IL1RA to facilitate establishment of the infection is innovative. Based on sequence homology to the Yersinia T3SS and its effector Ycs-Yop³, without wishing to be bound by theory, bteA not only increases IL1RA directly but also, interacts with host kinases, such as RhoGTPases, to manipulate host signals to promote inflammasome activation and subsequently increase IL1RA. Currently, there is no a mechanistic understanding on how bteA promotes cytotoxicity and cell death. Unraveling the molecular mechanisms that drive cell death upon injection of bteA will be critical for a better knowledge of the mechanisms by which the T3SS in Bordetella spp. manipulate host immune responses. In fact, previous data, by us and others, have indicated that RB50 promotes inflammasome activation^{16,20} and the bteA effector can be one of the contributing factors for this activation. Our finding can expand the role of bteA from being primarily cytotoxic^{4,21} to a more modulatory, immunosuppressive function. Importantly, differences in the cytotoxicity between bteA of BB and BP have been acknowledged, and this is due to one alanine insertion at the 503 position⁴. Our studies combining both strains BB/BP and mutants, BB harboring bteA from BP and viceversa, will also provide an insight in the role of that highly conserved Ala503 insertion in BP during the role of bteA and the Bordetella T3SS during the evolutionary process. Without wishing to be bound by theory, this insertion although correlates with a decrease cytotoxicity, which can translate in decrease inflammasome activation, can not decrease induction of IL1RA. Furthermore, IL1RA is already used as a therapy for autoinflammatory disorders, and it has been considered as a target for cancer treatments. Our findings can point to IL1RA as a potential target for treating bacterial infections and disease such as whooping cough, but also other respiratory diseases caused by bacteria and viruses.

The generation of a btrS-null mutant, which is defective for manipulating host immune responses¹⁶, in conjunction with the well-established BB murine model of respiratory infection, provides a unique and innovative system to study the role of epithelial²²⁻²⁴ and other immune^{10,17} cells in the establishment and length of infection. Moreover, as we combine BB and BP T3SS studies, this will also allow us to investigate the contribution of specific mutations (Ala503) during Bordetella spp. evolution. We have the technology established to genetically modify BB and BP to aid our studies¹⁷. We have created several mutant strains that have allowed us to generate preliminary data as well as to continue to develop this proposal, including but not limited to BB and BP knockout of bteA, bopN, bscN, double mutant bteA and bopN, the complemented strains, a BB strain that expresses bteA from BP a bteA-TEM reporter strain²⁵, and the constructions have also been done in BP. These reporter strains will allow us to combine flow cytometry and super-resolution microscopic techniques to study at the molecular and cellular level the role of bteA in the manipulation of the host cell. We will use several mice strains, including but not limited to Balb/c and C57BL/6J, IL1rn^{-/-26} (lack the gene encoding for IL-1 receptor antagonist), NLRP3 and Caspase-1 knockouts, as well as other conditional knockout strains that will be used in the context of bacterial infections (Jackson Laboratories). Combining wildtype/knockout mice, antibody depletion, and mutant bacterial strains (BB and BP), we will have an exclusive, highly innovative system to decipher the role of bteA in a clinically relevant disease model that allows for the first time to decipher the mechanisms enabling early establishment of bacterial lung infection and long-term lung persistence.

### C. Results

btrS Regulates an Immunosuppressive Pathway that Includes the T3SS.

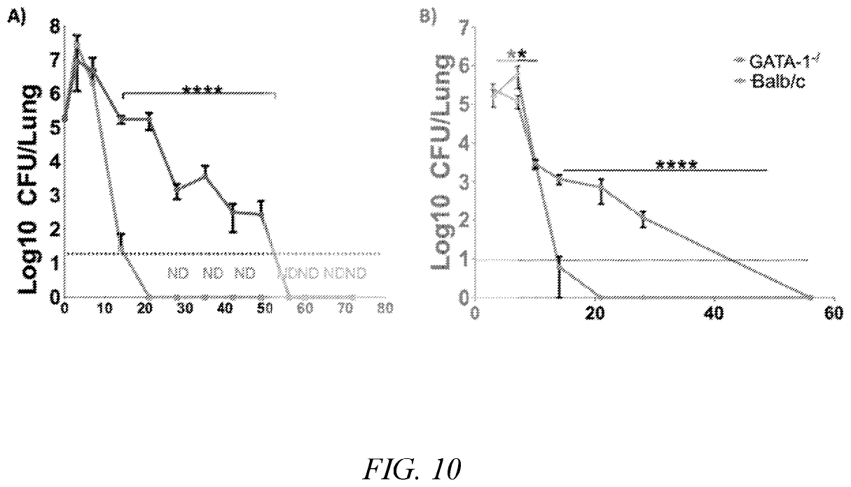

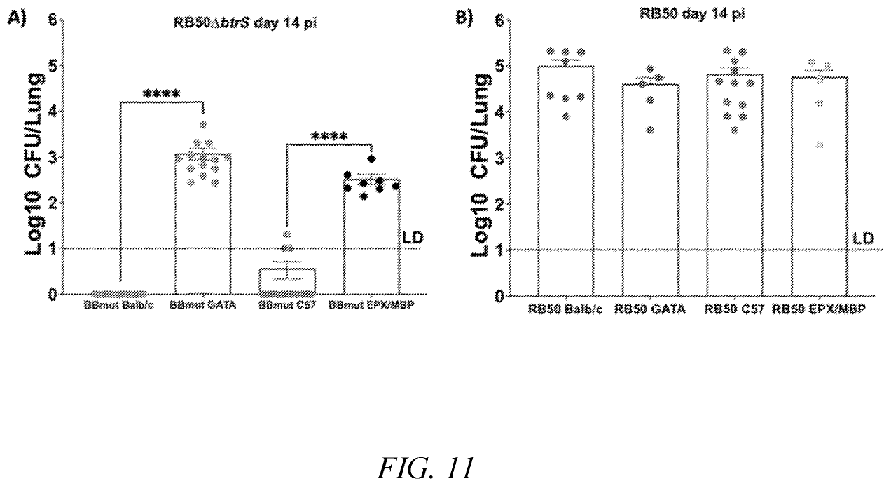

We wanted to better understand the molecular mechanisms that allow Bordetella spp. to counter host inflammatory signals¹⁰. We incubated B. bronchiseptica (RB50) with blood and serum to determine the genes that were differentially regulated in response to inflammatory signals contained in blood and serum¹⁵. Our results revealed that 89 genes were exclusively up-regulated in RB50, including several known toxins such as the type 3 secretion system (T3SS) utilized by the bacteria to suppress host immune responses^{3,10,27-30}. This differential gene expression in response to serum was highly conserved amongst the 3 classical Bordetella spp. including the human pathogen B. pertussis¹⁵. When investigating the regulatory pathways involved in this responses, we identified a bacterial sigma factor, btrS¹⁵ (also known as brpL)^{14,31}, that was upregulated 6-, 3-, and 2.5-fold upon incubation in blood, serum, and after internalization by macrophages, respectivelyl^{6,32}. Amongst the genes regulated by this sigma factor is the T3SS^{14,16}, which also was upregulated upon incubation with blood/serum. T3SS is one of the most effective bacterial immuno-suppressive mechanisms known to date, which functions by injecting toxins into the host cells to promote cell death^{3,4} and anti-inflammatory responses⁶. Because btrS regulates several bacterial immunosuppressive genes and it is highly conserved not only amongst Bordetella spp, but also in other bacterial species, without wishing to be bound by theory, btrS is a regulator of an immunomodulatory pathway^{14,16,33}. We deleted btrS from the genetic background of RB50, creating the mutant strain RB50ΔbtrS, named BBmut herein. We intranasally inoculated C57BL/6J mice to enumerate bacterial burden and lymphocyte populations in the respiratory tract using flow cytometry. While the wildtype RB50 strain persisted up to 56 days, BBmut was rapidly cleared by day 14 post-infection (FIG. 10, panel A) from the lung, cleared more rapidly from the trachea and, even more importantly, it also cleared from the nasal cavity⁴. Complementation of the mutation⁴ resulted in persistent infection¹⁶. When investigating the immune response to RB50 and BBmut, our results revealed that B and T cells (CD4 and CD8) are rapidly recruited to the site of infection following intranasal challenge with the BBmut but not the parent RB5016. Importantly, BBmut infection generates a robust immune response that provides long-lasting Th1/Th17 immunity against the 3 classical Bordetella spp.^{4,34}. These results indicate that btrS is essential for suppressing an effective, robust protective immunity against three classical Bordetella spp.^{16,34}.

### BBmut Requires Eosinophil Recruitment for Early Clearance of the Infection.

To interrogate what immune cell populations are suppressed during RB50 infection and are critical for rapid clearance of BBmut infection¹⁶, we followed the recruitment of immune cells using flow cytometry after infection with RB50 and BBmut. Significantly higher numbers of eosinophils were observed on days 3 and 7 post-infection in mice infected with BBmut compared to naïve and RB50 infected mice preceding the increase in B and T cells in the lungs¹⁷. To further validate the role of eosinophils in the immune response to RB50 and BBmut infection, we decided to use ΔdblGATA-1 mice¹⁷, herein called GATA, which are ablated of the eosinophil population and BALB/c, the genetic background of these mice (FIG. 10 panel B). We also used a second model of C57BL/6J and the mutant EPX/MBP^{-/-} mice, which specifically ablates the eosinophil population³⁵. While BBmut was practically cleared at day 14 post challenge of both wildtype mice, both eosinophil-deficient mice had increased bacterial burden in the lungs (FIG. 11, panel A). Interestingly, when the 4 mouse strains were challenged with the RB50 strain, no differences were found in persistence, indicating that the role of eosinophils is masked during RB50 infection (FIG. 11, panel B). It is important to note that eosinophil deficient mice challenged with BBmut had lower bacterial burden in the lungs at day 14 post-infection, indicating that eosinophils play a role in the rapid clearance of BBmut, but they are not the only driving force in this rapid clearance. Since our description of eosinophils contribution to Bordetella spp. immune responses, other investigators have continued to explore the link between eosinophils, asthma, allergies and Bordetella^{18,19} Overall, our results point to an essential role of eosinophils during the clearance of BBmut infection, indicating that eosinophils can play an important role during the generation of adaptive immune responses.

### RB50 Blocks Eosinophilic Pro-Inflammatory Responses.

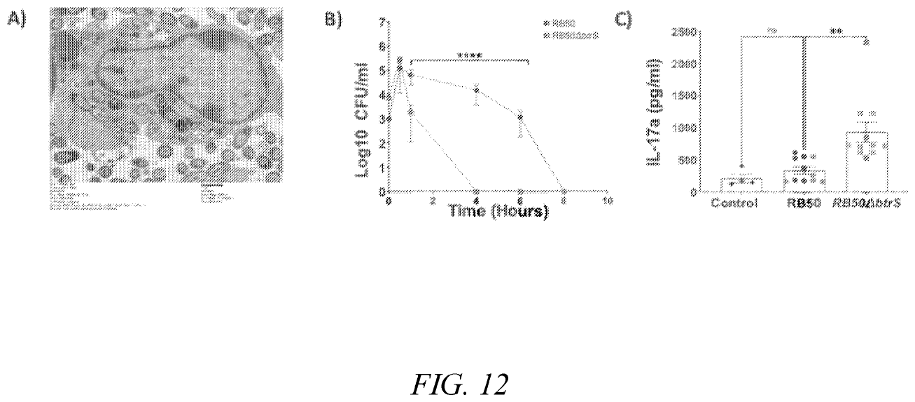

Our results indicated that eosinophils promote adaptive immune responses¹⁷ during BBmut infection but not RB50 (FIG. 11). Previous literature revealed that during gut infections with Helicobacter pylori, eosinophils promote long-term persistence³⁶. We wanted to investigate if RB50 can utilize eosinophils to promote anti-inflammatory environments that can facilitate persistence, similar to H. pylori³⁶. To investigate the interactions between eosinophils and these two bacterial strains (RB50 and BBmut) we used a minimalistic approach. We differentiated bone marrow progenitor cells into eosinophils using a protocol generously shared by Dr. Helene Rosenberg³⁷. After confirming >90% differentiation with flow cytometry following established protocols that used CD11b⁺SiglecF^{Hi} as gating strategy³⁷, cells were challenged with RB50 or BBmut to evaluate several eosinophil effector functions. Our results revealed that at a multiplicity of infection (MOI) of 10, eosinophils efficiently phagocytosed RB50 (FIG. 12, panel A) and BBmut (dns, n=3×3). When evaluating the ability of eosinophils to kill the bacteria, we found that at MOI=0.1 of BBmut (red) was completely killed by four hours post-infection, however, RB50 (blue) was not completely cleared until 8 hours post-infection (FIG. 12, panel B). We then evaluated the cytokines secreted after challenge at 4 hours post-infection at an MOI of 10 and defined the cytokine secretome in response to RB50 or BBmut. Our results revealed that eosinophils challenged with BBmut (red) secrete significantly more IL2 (dns, n=8×2) and IL17 (FIG. 12, panel C), indicating that eosinophils can phagocytose and kill Bordetella as well as act as pro-inflammatory immune cells that participate in immune responses to BBmut. Overall, these results indicate that RB50 suppress secretion of IL17, and eosinophil mediated killing by an unknown mechanism.

### RB50 Promotes IL1RA Expression In Vitro.

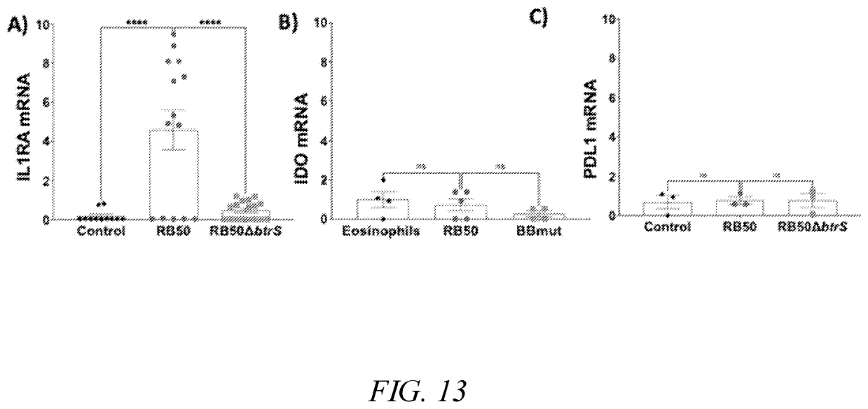

Our previous results demonstrate that eosinophils can secrete pro-inflammatory cytokines that stimulate Th1/Th17 responses in the presence of BBmut but not RB50. It is well-established that eosinophils can promote anti-inflammatory responses by secreting IL1RA³⁸, PD-L1³⁶, and IDO³⁹. In fact, during H. pylori infection, eosinophils secrete PD-L1 to dampen proinflammatory responses³⁶. Without wishing to be bound by theory, similarly, RB50 promotes the expression of at least one of these molecules to dampen proinflammatory responses and facilitate lung infection. Using bone marrow-derived eosinophils challenged at an MOI of 10 with RPMI (black), RB50 (blue), or BBmut (red), we found that infection with RB50 but not BBmut promotes expression of IL1RA by eosinophils (FIG. 13, panel A), while no changes in the expression of IDO (FIG. 13, panel B) and PD-L1 (FIG. 12, panel C) were detected. Overall, these results indicate that RB50 promotes IL1RA expression in eosinophils, which is associated with anti-inflammatory responses.

### RB50 Achieves Persistent Infection Through IL1RA Induction.

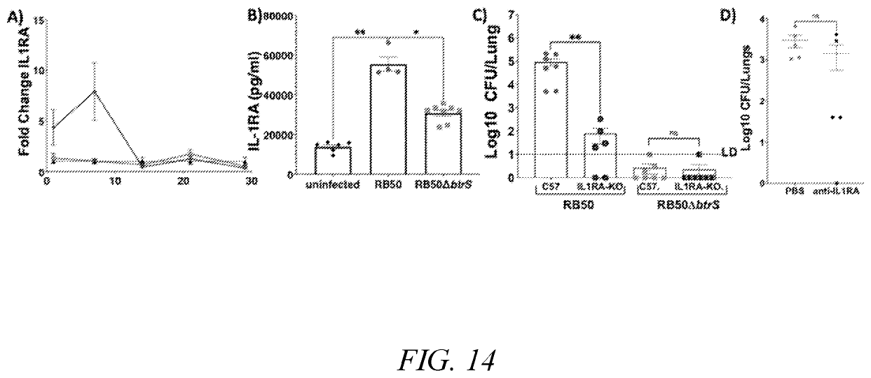

It has been noted that IL1RA has a role in aggravating infections⁴⁰, one example being Staphylococcus aureus septicemia, where pathology is increased when mice are treated with IL1RA⁴¹. To evaluate the effects of IL1RA in vivo, we challenged mice with PBS (black), RB50 (blue), or BBmut (red) and extracted bulk RNA from the lungs at different time points. We found that IL1RA mRNA increases as early as day 1 post-infection, peaking at day 7 and then returning to basal levels at day 14 after infection with RB50 (blue) (FIG. 14, panel A), similar to the trend previously shown during P. aeruginosa lung infection⁴². We also observed increased protein levels at day 7 post-infection in the lungs of mice challenged with RB50 (blue) but not as much with BBmut (red) (FIG. 14, panel B). When investigating the role of IL1RA in persistence, we found that intraperitoneal supplementation of IL1RA increases bacterial burden during infection with RB50 and BBmut (dns, n=4-8), accordingly with previously published data for other infections⁴¹. To determine the effects of IL1RA during infection with RB50 and BBmut, we used a knockout mouse (Ilr1n^{-/-}) (FIG. 14, panel C) and our results show that RB50 (blue with black shadow) bacterial burden is significantly decreased in the absence of IL1RA. Depletion of IL1RA with antibodies resulted in more rapid clearance of RB50 (dns, n=8 mice) and although not significant, in 3 of the 5 mice infected with B. parapertussis, the bacterial burdens in the lungs was reduced (FIG. 14, panel D). Overall, these results indicate that IL1RA promotes bacterial persistence in the lungs, and ablation of IL1RA using a knockout mouse model or antibodies anti-IL1RA results in more rapid clearance of RB50.

The Bacterial Type 3 Secretion System (T3SS) Effector, bteA, is Required for IL1RA Induction.

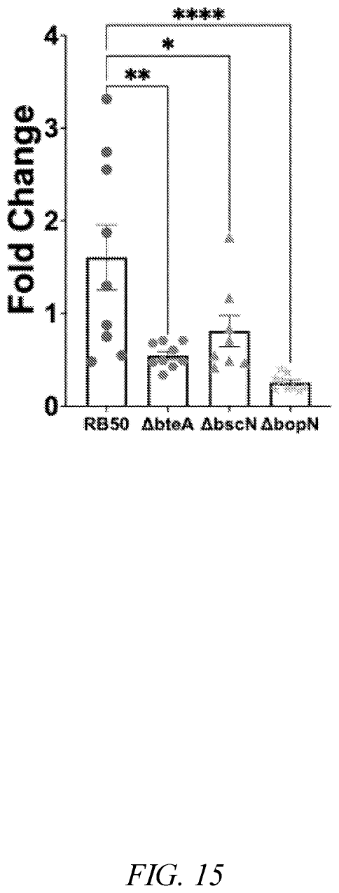

One of the virulence factors regulated by btrS is the T3SS indicating a T3SS effector can promote IL1RA expression. It has been shown that the T3SS promotes long term infections by BB⁶ and BP⁷. Indeed, mice infected with the RB50□bscN mutant, which does not have a functional T3SS due to the absence of the ATPase, did not have significantly increased levels of IL1RA in the lungs compared to untreated controls. We then investigated the T3SS effectors, bteA which causes cytotoxicity by an unknown mechanism, and bopN, which facilitates bteA translocation. Infection with knockouts of the effectors bteA and bopN, respectively, resulted in lower IL1RA mRNA levels (FIG. 15). While bteA is delivered to target cells, bopN is required for the translocation of bteA, indicating that bteA is responsible for the IL1RA induction. We confirmed at the protein level (n=1) this reduction. We are now testing these results on the human strain BP and performing the corresponding controls of complemented BB strains. We also created a BB strain that harbors the bteA of BP and a BP strain that harbors the bteA of BB. Overall, our results indicate that bteA, once injected via T3SS, promotes expression and induction of IL1RA in the lungs.

### Epithelial Cells Rapidly Secrete IL1RA in Early Stages to Facilitate Infection.

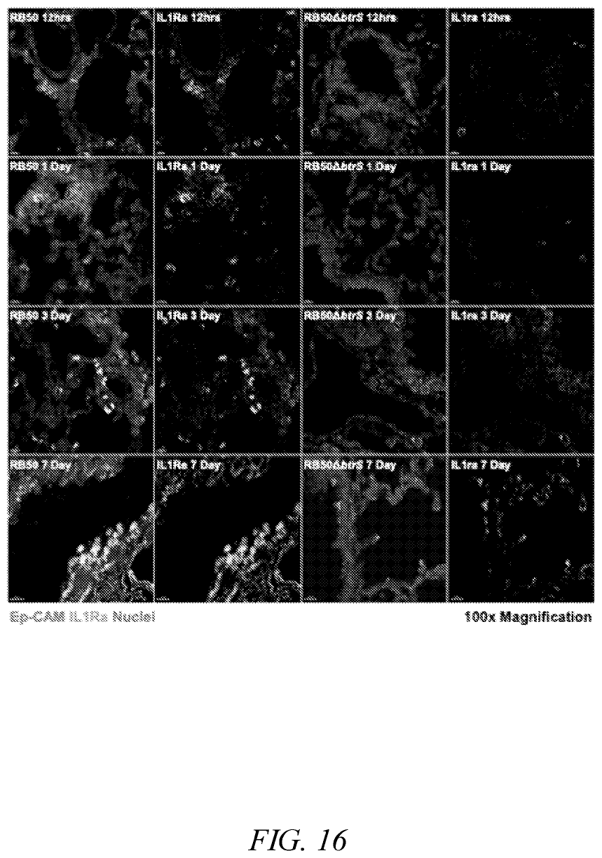

Based on our results we wanted to investigate when and how IL1RA secretion started. Without wishing to be bound by theory, the first cells that are in contact with the bacteria are lung epithelial cells and that they can start the anti-inflammatory response that promote initial establishment of the infection. We performed qRT-PCR (FIG. 13) and immune-fluorescence staining (FIG. 16) of lungs at different times post-infection. Our qRT-PCR data revealed an increase of IL1RA mRNA level as early as day 1 post-infection, peaking at day 7 and returning to basal levels at day 14 post-infection. Our IL1RA-specific microscopy results also confirmed the RNA data and provided a clearer understanding. Our photos (n=1-2) show that after 12 hours post-infection with RB50, levels of IL1RA already increase. Interestingly, the signal show patches of IL1RA across the section, indicating that these can be areas where the bacteria are in contact with those epithelial cells. We can also see how the levels of IL1RA increase to a maximum intensity at day 7, where the IL1RA signal is more spread in around the lung. In our BBmut infected mice, although there is an increase in signal, this is not reaching the same intensity as the RB50 infected mice. Moreover, these images also show that at these early times, most of the secretion of IL1RA occurs in epithelial cells, which have been reported as higher producers of IL1RA together with eosinophils³⁸.

### Model.

Based on these results, we provide the following working model (FIG. 17): Upon infection, Bordetella spp. injects bteA via T3SS into lung epithelial cells. bteA promotes the transcription and secretion of IL1RA that in turn dampens proinflammatory responses and immune cell recruitment allowing for the initial establishment of infection. Because it is known that the bteA effector is cytotoxic^{4,21}, without wishing to be bound by theory, in a dose-time dependent manner, RB50, via bteA effector, promotes inflammasome activation¹⁶. The functional and sequence homology to the Yersinia yop effectors³, which have been shown to directly interact with RhoGTPases, indicate this model and based on this we provide a similar mode of action; where bteA interferes with RhoGTPases promoting inflammasome activation. As inflammasome activation associates with an increase in IL1β and Il1RA⁴², without wishing to be bound by theory, this can also be responsible for the increase in IL1RA. As we also observe an increase of IL1RA expression in eosinophils, in vitro, in response to RB50 infection in vitro, once bacteria cross the epithelia, eosinophils are amongst the next target for bteA delivery, which enhances the anti-inflammatory responses in the lungs in a similar way they do in the guts during H. pylori infection (FIG. 17).

Taken together, our identification of btrS and its knockout in the RB50 background has allowed for us to identify immunosuppressive pathways regulated by btrS that reveals the importance of the Bordetella spp. T3SS effector bteA, and IL1RA. Moreover, our data indicate that epithelial cells can be critical during early stages of colonization/infection. Overall, completion of the proposal will provide us with a detailed mechanistic understanding of how BB/BP utilize the T3SS effector, bteA. to promote IL1RA induction to allow for initial colonization and long-term persistent infection. This knowledge can guide the development of therapies and reduce the disease burden caused by related human strains.

### D. Approach

Rationale: By deleting the sigma factor-encoding btrS gene from BB, we uncovered an essential role of eosinophils in mounting an effective and sterilizing immune response to BB infection¹⁷. In fact, we have found evidence that indicate that Bordetella spp. can block eosinophil effector functions to promote long-term persistence. Our data indicate that the wildtype B. bronchiseptica strain, RB50, but not BBmut promote anti-inflammatory responses from epithelial cells in vivo, and eosinophils in vitro, which can facilitate early events of colonization, infection (FIG. 17), by inducing the expression and secretion of the anti-inflammatory molecule IL1RA. We show that upon initial contact with epithelial cells IL1RA is being induced by bteA injection via the T3SS. Once the bacteria cross the epithelial barrier, eosinophils and other immune cells will be targets of bteA injection which will enhance IL1RA secretion and anti-inflammatory responses (FIG. 17). We further provide that the mechanism includes bteA interaction with RhoGTPases, which leads to inflammasome activation, increased IL1β secretion and IL1RA induction. Associated with that is inflammasome mediated cell death (FIG. 18).

Herein, we plan to follow up on these exciting observations to understand how BB/BP promote the expression of IL1RA and inflammasome activation via bteA. Our overarching working model is that BB/BP utilize the T3SS effector, bteA, to promote IL1RA expression allowing for initial colonization and long-term persistent infection (FIG. 17 and FIG. 18). Overall, this proposal will investigate the molecular mechanism by which bacterial pathogens successfully utilize the T3SS to first establish infection and allow them to persist within the host.

### E. Rigor and Reproducibility

Bacterial strains were sequenced, and several frozen stocks were made to make sure no mutations are being acquired. Experiments performed in the lab are registered in log files containing the information and detailed protocols performed for each experiment and raw and analyzed data. Experiments are coded with a log number. In vitro experiments will be performed at least in 5 independent biological replicates and 3 technical replicates (n=3×3), which will help us ascertain reproducibility. Animal infection experiments will be independently repeated twice using 5-6 animals per infection, time point and group following our calculations using Russ Lenth's power and sample size calculator to determine the number of mice required for p<0.05 with a standard deviation of 20% (control versus challenged with the different pathogens). Infection and organ collection will be blinded using a numerical code to ensure unbiased analyses. We have frozen inoculums to make sure that the inoculum that mice receive is always the same (we have at the moment 25-50 inoculums of each strain included in this grant). When assessing the function of a gene/pathway, we will use knockout animals combined with inhibitors or antibodies for depletion to have two independent models. For flow cytometry, experiments will be run in parallel with single stains and Fluorescence minus one. Samples for flow cytometry are provided to the immunophenotyping core as single cell suspensions or supernatant in a blinded manner. We will use a panel of markers for epithelial cells, eosinophils, and other immune cells that are based on a combination of markers and not only one, due to the overlap of some of the markers with other immune cell populations. To evaluate gene expression, we will be using qRT-PCR with NTC controls, validation of primers, and the adequate positive and negative control for the qRT-PCR. For phosphorylation studies, we will stimulate with growth factors as positive controls. To evaluate microscopic analyses, isotype controls and unstained controls will be included in each experiment. Finally, we are using BB and a clinical strain of BP from a recent isolate. However, our most relevant findings will also be studied on circulating B. pertussis and B. bronchiseptica strains to corroborate our results using human pathogens provided by the CDC.

### F. Approach

SA1: Investigate the Molecular Mechanisms by which the bteA Effector Promote Early Events of Colonization.

In vitro data revealed that RB50 promotes the expression of IL1RA in eosinophils mediated by the T3SS effector, bteA. We are currently investigating the effects of bteA of BP, however, previous published data indicate that bteA encoded by BP also allows for colonization⁷ and cell death⁴. Our results indicate that IL1RA mediates persistence but interestingly, our in vivo data shows that epithelial cells secrete IL1RA in response to RB50 infection as early as 12 hours post-infection. Taken together, these results indicate that, upon infection, lung epithelial cells, the first cells to come in contact with Bordetella spp., will be injected with the bteA effector, which induces IL1RA expression facilitating early establishment of the infection. Once the bacteria cross the epithelial barrier, other immune cells, including eosinophils; as our in vitro data indicate, will contribute to the anti-inflammatory environment in the lungs that facilitates long-term persistence. We provide the following aims:

SA1.1. Evaluate IL1RA Responses to bteA in a Dose and Time Dependent Fashion Using Lung Epithelial Cells.

Here, we will use in vitro experiments to evaluate the time and dose response of epithelial cells to bteA injection. We will use BB and BP that have a lactamase bteA reporter system (bteATEM-reporter) and as negative control the knockout bteA respective mutants. We will infect A549 lung epithelial cells with the aforementioned bacterial strains at MOI of 0,1; 1; 10. As controls serve media alone and RB50. At 0.5, 1, 2, 4, 6, and 8 hours post-infection, we will execute different experiments. 1) We will perform immunofluorescence microscopy (IF) to colocalize the bacteria, cells injected with bteA, and IL1RA. We will use RNAscope combined with IF to determine colocalization and semiquantitative levels of il1ra mRNA and IL1RA protein. This will allow us to identify the foci of IL1RA expression and secretion following injection of bteA. 2) Following infection, we will collect the cells to extract RNA and subject it to qRT-PCR to evaluate at the transcriptional level of expression of bteA and IL1RA. We will also quantify other T3SS genes as control. We will measure recA and β-actin as bacterial and host cell housekeeping mRNA for normalization purposes. This experiment will complement the RNAscope and allow direct quantitative evaluation of the effects of bteA on IL1RA expression. 3) We will use the supernatant of the previous experiments to perform IL1RA ELISA to quantify the effects of bteA on IL1RA secretion at a protein level.

TABLE 2
Current innate panel for flow cytometry used in our studies.

| Marker/Fluorophore | Company | Catalog No. | Final Dilution |
|---|---|---|---|
| Zombie Yellow | Biolegend | 423103 | 1:500 (2 ul |
| | | | in 1.0 ml) |
| CD45 BV510 | Biolegend | 103137 | 1:400 |
| CD11b AF488 | Biolegend | 101217 | 1:2000 |
| Ly6G BV711 | Biolegend | 127643 | 1:200 |
| F4/80 AF700 | Biolegend | 123129 | 1:100 |
| CD193 BV421 | Biolegend | 144517 | 1:200 |
| Siglec F PE | Biolegend | 155505 | 1:200 |
| CD11c PerCP-Cy5.5 | Biolegend | 117328 | 1:400 |
| Ly6C PE/Cy7 | Biolegend | 128017 | 1:800 |
| MHCII BV605 | Biolegend | 107639 | 1:400 |
| CD80 APC | Biolegend | 104714 | 1:100 |
| CD86 BV785 | Biolegend | 105043 | 1:100 |

SA 1.2. Identify the bteA Target Cells in a Spatiotemporal Manner In Vivo.

Here, we will identify the cells injected with the T3SS effector, bteA, in a spatiotemporal manner in vivo. We will intranasally inoculate mice with PBS, BB/BP with a bteATEM-repoter²⁵, BB/BPΔbteA. At 12 hours, 1-, 3-, 7-, 14-, and 28-days post-infection, mice will be euthanized to perform the following experiments. 1) Time course of infection by enumerating CFUs from nasal cavity, trachea and lungs^{16,17}. This will allow us to investigate the impact of bteA on colonization and persistence in the respiratory tract, which is not fully understood yet. Currently, we are planning a time course up to 28 days but we will consider later times if we start to see slight differences. 2) Flow cytometry studies. Lungs will be collected to generate a single cell. Cells will be stained with CCF4-AM (Invitrogen for the bteATEM-reporter) and our current innate panel (Table 2) as starting point. We will adjust our panel if indicated. We will then sort the cells for positive/negative for bteA. From those we will gate for the different innate immune cell populations (cd11c+, cd11b+F4/80+, cd11b+SiglecF+, cd11b+Ly6G+) and epithelial cells (EpCAM=CD326). This will allow us to identify which and what percentage of cells are injected with bteA at different times post-infection. The procedures are already well established in the lab and the core. 3) Immunofluorescence staining (IF). We will collect lungs and following intratracheal perfusion with PBS and 4% PFA, we will embed the tissues in paraffin to proceed with the sectioning (this technique is well established in the lab). We will subject tissue section to IF analyzing at least 3 lung sections per mouse. For the staining we will use EpCAM (BioLegend, #118206) and CCF4-AM (Invitrogen for the bteATEM-reporter). These experiments will be quantified with the Keyence software and will be performed with the help of Dr. Sapp (see letter of support). This allows us to identify the cells that produce IL1RA at different post-infection times in a spatial manner. We will be able to identify if the IL1RA production starts as foci and then spreads throughout the lungs.

SA 1.3. Investigate the Effects of bteA on IL1RA Induction In Vivo.

Following the same experimental setting as SA1.2. We will; 1) Perform the same flow cytometry studies as shown in 1.2, however, once we have the positive/negative bteA cells, we will see how many of those are also IL1RA+. This will allow us to identify the contribution of bteA+ and bteA-cells to the total IL1RA response; 2) collect the lungs for IF staining. For the staining we will use the two antibodies previously provided in 1.2 and IL1RA (for protein R&D, #BAF480, for RNA Scope ACD, #495101). These will allow us not only to see how much IL1RA are epithelial cells producing, but also the location where the IL1RA is being produced.

### SA1. Prospective Results and Alternatives

### Prospective Results—

Without wishing to be bound by theory, we will see epithelial cells produce IL1RA in response to bteA injection in a dose dependent manner both in vitro and in vivo. Epithelial cells will be the first cells targeted by the T3SS. However, at later time points the bacteria will have had enough time to cross the epithelial barrier and other cells, such as eosinophils, will be contributing to the increase in IL1RA (FIG. 17). We based this working model on our results, (FIG. 16) where we see first patches of IL1RA in epithelial cells as quickly as 12 hours post-infection and these patches increase in size gradually until day 7 when the whole epithelia and areas inside the lungs express IL1RA. We also predict that mostly bteA+ cells will express IL1RA.

SA2: Define the Role of bteA in Inflammasome Activation.

RB50 promotes NLRP3 mediated inflammasome activation^{16,44} in different cell populations. However, the bacterial mechanism that triggers this activation is not fully understood. Our data indicate that inflammasome activation is a btrS-mediated mechanism¹⁶. Previous literature has shown that in P. aeruginosa, IL1RA induction correlates with NLRP3 activation⁴², indicating that maybe the T3SS of Bordetella spp., which is btrS regulated, can be responsible for the inflammasome activation and subsequent IL1RA increase. The T3SS of Bordetella spp, and its effectors harbor structural, homology, and functional similarities with the Yersinia spp³. T3SS, and it has been published that Yersinia spp. effectors induce inflammasome activation⁴⁵. Based on these published data and our findings, without wishing to be bound by theory, bteA promotes inflammasome activation leading to an increase in IL1RA levels.

SA2.1. Evaluate the Role of bteA on Inflammasome Activation In Vitro.

bteA is cytotoxic⁴, however, the mechanism by which cells die is not known. Epithelial cells⁴⁴ and macrophages¹⁶ have been shown to activate inflammasome and secrete IL1β in response to Bordetella spp. infection indicating that inflammasome can be total/partially responsible for the cell death following bteA injection. Also, IL1β is known to trigger IL1RA expression⁴². The Yersinia spp, and Bordetella spp. T3SS are highly conserved³ and Yersinia spp. T3SS effectors cause inflammasome activation and an increase in IL1β45. We show that bteA induced cell death is triggered by inflammasome activation. We will perform in vitro studies using lung epithelial cells, A549, and eosinophils differentiated from bone marrow progenitors³⁷. The following assays will be employed: 1) Qrt-PCR analysis. At a confluency ≥85%, we will challenge cells with media alone, the wildtype BB/BP, bteA-null mutants, and complemented strains at MOI 0, 1; 1; and 10. At 1-, 2-, 4-, and 8-hours post-infection, we will isolate RNA and quantify NLRP3, NLRC4, IL1β, and Caspase-1 Mrna levels by Qrt-PCR using β-actin as housekeeping gene. In a follow up experiment, we will use the reporter strains and sort cells for bteA prior to RNA extraction. These experiments will allow us to evaluate the contribution of bteA to the transcriptional activation of different inflammasome markers in a time-dose dependent manner. 2) IF staining. We will infect A549 in a 96 well plate format. We will fix the samples at each time point and perform immunofluorescence assays. For this assay we will replace the wildtype strains by the bteATEM-reporter to identify bteA+ and bteA-cells. We will stain CCF4-AM (substrate for bteA), NLRP3 (ThermoFisher, #768319), NLRC4 (ThermoFisher, #88997), and Caspase-1 (ThermoFisher, #32909). We will image the 96 well plate assay with the cellomics ArrayScan V, automated fluorescent microscopic imaging system that also perform an unbiased analysis. This will allow us to identify changes at the protein level of different inflammasome markers and colocalize those with bteA+ cells. Moreover, this will provide a spatiotemporal understanding of cell death following bteA injection in an unbiassed manner. We will also monitor Caspase-1 activation by Western blot. 3) Real time apoptosis. We will use the ApoTracker staining (BioLegend #Apo-15) to monitor in real time cell apoptosis following infection with each bacterial strain. This assay will be run on a TECAN Spark and will provide an unbiases quantitative data of apoptosis in real time. 4) ELISA. Finally, we will use the supernatants from previous experiments to evaluate levels of IL1β and IL1RA via ELISA, and Caspase-1 activation (Caspase-1-Glo, Promega¹⁶). Without wishing to be bound by theory, we will see that wildtype bacteria induce an increase in the levels of these markers.

SA2.3. Determine the Effects of bteA on Inflammasome Activation In Vivo.

Based on our previous results¹⁶ and literature^{4,42}, without wishing to be bound by theory, bteA injection into host cells will activate the inflammasome pathway leading to cell death. To evaluate the effects of bteA on inflammasome mediated cell death in vivo, we will intranasally inoculate mice with PBS or PBS containing BB/BP bteATEM-reporter, bteA knockouts, and bteA complemented strains. At different times post-inoculation (day 3, 7, 14, 21, and 28), we will perform the following assays. 1) Flow cytometry to identify the cells injected with bteA and evaluate caspase-1 activation. We will use the strategy outlined in SA1.2 and include caspase-1 (Abcam, #ab219935) and apoptosis (BioLegend, Apo-15) markers. We will gate first for bteA+ and bteA-cells. This aim will allow us to determine what cells were injected and which ones are undergoing apoptosis as well as the percentage of those that are bteA+. 2) IF staining. We will fix the lungs and use 3 sections per mouse to perform IF combined with RNA scope to evaluate levels of expression of NLRP3, NLRC4, Caspase-1, IL1RA in bteA+ cells. We will use the antibodies stated in aim 1.2 as well as from 2.1 to be able to detect the areas of the lung that are undergoing apoptosis following injection of bteA. This will provide us spatial information, and at early times only epithelial cells injected with bteA will undergo inflammasome activation. Cell death will also increase the levels of IL1RA secreted by neighboring cells by paracrine mechanisms. 3) ELISA. We will use supernatant of lung homogenates collected in PBS containing protease inhibitor to evaluate levels of IL1β, IL18, and IL1RA of the different study groups at different times post-infection. This will allow us to determine the contribution of bteA to cytokine secretion (inflammasome related cytokines).

### SA2.3. Determine the Effects of Inflammasome Activation on IL1RA Induction.

To identify the role of inflammasome activation on IL1RA induction, we will combine the use of knockout mice (Casp-1 tm1Flv/J, NLRP3^{-/-} mice, NAIP-NLRC4 deficient mice) with inhibitors of caspase-1 (Ac-YVAD-cmk, InvivoGen), and NLRP3 (CY09, medkoo). To evaluate the individual roles of the different inflammasome components in IL1RA induction, we will intranasally inoculate mice with PBS or PBS containing BB/BP wildtype, bteA knockout, bteA complemented and bteA reporter. At 3-, 7-, and 14-days post-infection, we will perform the experiments described in SA2.2; 1) flow cytometry to identify cells injected with bteA and evaluate caspase-1 activation, 2) perform IF combined with RNA scope to evaluate levels of expression of NLRP3, NLRC4, Caspase-1, and IL1RA in bteA⁺ cells, and 3) use supernatant of lung homogenized to evaluate levels of IL1β, IL18, and IL1RA on the different study groups.

### SA2. Prospective Results and Alternatives

### Prospective Results—

We know that bteA from Bordetella spp. is cytotoxic⁴ and other bacteria use similar T3SS^{45,46} to promote inflammasome, so it can make sense that bteA cause cell death using similar mechanisms as those shown for Yersinia spp.^{45,46} Without wishing to be bound by theory, in vitro, we will observe activation of inflammasome following bteA injection (FIG. 18). The in vivo data will further support this, and moreover will be able to detect that the cell death caused by bteA start in the outer layers of the lung, epithelial cells, and progress to the internal parts of the lung with the progression of the disease (FIG. 17). The addition of the knockout animal models will further allow us to demonstrate, following Koch's postulates, the role of bteA in inflammasome activation.

### SA3: Identify the Interactions Between BteA and Host Binding Partners.

Based on amino acid sequence homology with Yersinia spp.³ effectors^{45,46} and 3D structure homology with Clostridium toxins⁴⁷, BteA directly interacts with host signaling proteins like AKT kinases and RhoGTPases to disrupt cell signaling. Dr. Seema Mattoo has expertise in Bordetella pathogenesis^{48,49} as well as deciphering functions of new bacterial effector proteins^{50,51}. The work in this aim involves a combination of biochemical and cell biological approaches.

### SA3.1. Determine the Changes in Phosphorylation Profile Induced by BteA.

Activation of AKT, RhoGTPase⁴⁵, and pyrin^{45,46} is known to induce increase of IL1RA⁵². Using in vitro mammalian cells (A549 and RAW macrophages), we will assess the phosphoproteome of mammalian cells when exposed to BteA. Specifically, 1) BteA will be ectopically expressed in mammalian cells following transient expression of BteA under a mammalian CMV promoter; 2) cells will be dosed with various concentrations of affinity and SEC (size exclusion chromatography) purified bacterially expressed BteA; and 3) cells will be infected with Bordetella strains (BB/BP, bteA-null for both strains, and BB-expressing bteA BP, BP expressing BB-bteA and the complemented strains). Mammalian cells extracts from treated and control samples will be analyzed by western blotting with phosphor-specific antibodies targeting AKT as well as by taking an unbiases phosphoarray approach (Catalog No: PEX100, Full Moon Biosystems). Samples will also be analyzed by mass spectrometry (LCMS/MS). Target protein candidates with altered phosphorylation profiles will be confirmed with targeted western blot assays.

### SA3.2. Identification & Validation of BteA Binding Partners.

Results from SA3.1 will inform us of putative kinase pathways and binding partners for BteA. These candidates will be assessed for direct binding using pulldown assays of mammalian extracts run over BteA-H₆ bound to a cobalt column or by ectopically expressing FLAG-tagged BteA in mammalian cells. Candidate interactors will be confirmed by western blotting. Based on amino acid sequence homology³, 3D structures predictions⁴³ and preliminary studies, without wishing to be bound by theory, detecting AKT and RhoGTPases such as RhoA known to be involved in IL1RA activation. To avoid non-physiological interactions stemming from traditional pulldown assays, we will employ a proximity labeling approach to identify physiologically relevant targets of BteA. Specifically, APEX engineered peroxidase⁵³ tagged BteA will be expressed in mammalian cells. Proteins interacting with BteA will be biotinylated and isolated using streptavidin beads and identified via LCMS/MS, as previously done in the Mattoo lab⁵⁰. Therefore, these and other identified candidate interacting proteins will be cloned and bacterially expressed and purified, to test their binding kinetics for BteA using Biolayer Inferometry⁵⁴. The binding kinetics will provide insight into BteA's target specificity and potential mode of action.

### SA3.3. Binding Kinetics and Structural Modeling of BteA and Target Proteins.

Candidates identified above will be cloned and bacterially expressed and purified, to test their binding kinetics for BteA using Biolayer Inferometry, a technique routinely performed in the Mattoo lab⁵⁴ The binding kinetics will provide insight into BteA's target specificity and potential mode of action. Additionally, a partial crystal structure for BteA has been solved (PDB RGN). Combined with AlphaFold2.0 analysis, we will assess protein-protein interactions of putative binding partners with BteA to predict regions of BteA involved in binding. These will be tested by site directed mutagenesis and deletion of BteA-target interfaces and assessing binding affinities of the mutant BteA to the target. We predict such an analysis will help define BteA's mode of target recognition.

### SA3. Prospective Results and Alternatives

### Prospective Results—

Based on preliminary results and sequence similarity to know bacterial effectors, without wishing to be bound by theory, binding partners in the AKT and RhoGTPases signaling pathway causing the disruptions in cell signaling and inflammation. In the event we are wrong, our use of unbiased approaches like proximity labeling will allow us to identify other potential targets of BteA.

### G. Epilogue

This proposal will provide a better understanding of the role of the bteA effector on host immunosuppression. This is to our knowledge the first study that focuses on understanding the role of a T3SS effector in early events of colonization and persistence, and how both are mediated by IL1RA. While an immunosuppressive role for Bordetella's T3SS system has been predicted, to our knowledge, this proposal is the first assessment of a Type 3 secreted effector in Bordetella mediated suppression of host immune responses. As increase in IL1RA occurs after infection with other respiratory pathogens, our work has the potential to greatly impact the development of new therapeutics that can be used not only to treat Bordetella spp. infections but also other respiratory pathogens. Understanding how bacteria manipulate the host immune responses at the molecular level, to allow for colonization and long-term infection is essential for developing new vaccines and therapies. For example, our identification of IL1RA as an immunomodulatory factor in Bordetella disease makes it a promising target. Our findings cannot only impact Bordetella spp, but also other mucosal pathogens that use similar mechanisms to promote colonization and persistence by blocking eosinophil effector functions.

### REFERENCES CITED IN THIS EXAMPLE

1. Coburn B, Sekirov I, Finlay B B. Type III secretion systems and disease. Clin Microbiol Rev. October 2007; 20(4):535-49. doi: 10.1128/CMR.00013-07
2. Kendall M M. Extra! Extracellular Effector Delivery into Host Cells via the Type 3 Secretion System. MBio. May 2017; 8(3) doi: 10.1128/mBio.00594-17
3. Kamanova J. Type III Secretion Injectosome and Effector Proteins. Front Cell Infect Microbiol. 2020; 10:466. doi: 10.3389/fcimb.2020.00466
4 Bayram J, Malcova I, Sinkovec L, et al. Cytotoxicity of the effector protein BteA was attenuated in Bordetella pertussis by insertion of an alanine residue. PLoS Pathog. Augst 2020; 16(8):e1008512. doi: 10.1371/journal.ppat.1008512
5 Abe A, Nishimura R, Kuwae A. Bordetella effector BopN is translocated into host cells via its N-terminal residues. Microbiol Immunol. June 2017; 61(6):206-214. doi: 10.1111/1348-0421.12489
6. Nicholson T L, Brockmeier S L, Loving C L, Register K B, Kehrli M E, Shore S M. The Bordetella bronchiseptica type III secretion system is required for persistence and disease severity but not transmission in swine. Infect Immun. March 2014; 82(3):1092-103. doi: 10.1128/IAI.01115-13
7. Fennelly N K, Sisti F, Higgins S C, et al. Bordetella pertussis expresses a functional type III secretion system that subverts protective innate and adaptive immune responses. Infect Immun. March 2008; 76(3):1257-66. doi: 10.1128/IAI.00836-07
8. Warfel J M, Beren J, Merkel T J. Airborne transmission of Bordetella pertussis. J Infect Dis. September 2012; 206(6):902-6. doi: 10.1093/infdis/jis443
9. Kilgore P E, Salim A M, Zervos M J, Schmitt H J. Pertussis: Microbiology, Disease, Treatment, and Prevention. Clin Microbiol Rev. July 2016; 29(3):449-86. doi: 10.1128/CMR.00083-15
10. Gestal M C, Whitesides L T, Harvill E T. Integrated Signaling Pathways Mediate Bordetella Immunomodulation, Persistence, and Transmission. Trends Microbiol. February 2019; 27(2):118-130. doi: 10.1016/j.tim.2018.09.010
11. Linz B, Ivanov Y V, Preston A, et al. Acquisition and loss of virulence-associated factors during genome evolution and speciation in three clades of Bordetella species. BMC Genomics. September 2016; 17(1):767. doi: 10.1186/s12864-016-3112-5
12. Bouchez V, Guillot S, Landier A, et al. Evolution of. Euro Surveill. September 2021; 26(37)doi: 10.2807/1560-7917.E S.2021.26.37.2001213
13. Dewan K K, Linz B, DeRocco S E, Harvill E T. Acellular Pertussis Vaccine Components: Today and Tomorrow. Vaccines (Basel). May 2020; 8(2)doi: 10.3390/vaccines8020217
14. Ahuja U, Shokeen B, Cheng N, et al. Differential regulation of type III secretion and virulence genes in Bordetella pertussis and Bordetella bronchiseptica by a secreted anti-σ factor. Proc Natl Acad Sci USA. March 2016; 113(9):2341-8. doi: 10.1073/pnas. 1600320113
15. Gestal M C, Rivera I, Howard L K, et al. Blood or Serum Exposure Induce Global Transcriptional Changes, Altered Antigenic Profile, and Increased Cytotoxicity by Classical Bordetellae. Front Microbiol. 2018; 9:1969. doi: 10.3389/fmich.2018.01969
16. Gestal M C, Howard L K, Dewan K, et al. Enhancement of immune response against Bordetella spp. by disrupting immunomodulation. Sci Rep. December 2019; 9(1):20261. doi: 10.1038/s41598-019-56652-z
17 Gestal M C, Blas-Machado U, Johnson H M, et al. Disrupting. Microorganisms. November 2020; 8(11) doi: 10.3390/microorganisms8111808
18. Nguyen N T D, Pathak A K, Cattadori I M. Gastrointestinal helminths increase. Elife. Nov. 8, 2022; 11doi: 10.7554/eLife.70347
19. Belcher T, Ait-Yahia S, Solans L, et al. Live attenuated pertussis vaccine for prevention and treatment of allergic airway inflammation in mice. NPJ Vaccines. Jun. 23, 2022; 7(1):66. doi: 10.1038/s41541-022-00494-w
20. Elizagaray M L, Gomes MTR, Guimaraes E S, et al. Canonical and Non-canonical Inflammasome Activation by Outer Membrane Vesicles Derived From. Front Immunol. 2020; 11:1879. doi: 10.3389/fimmu.2020.01879
21. Guttman C, Davidov G, Shaked H, et al. Characterization of the N-terminal domain of BteA: a Bordetella type III secreted cytotoxic effector. PLoS One. 2013; 8(1):e55650. doi: 10.1371/journal.pone.0055650
22. Lamberti Y, Gorgojo J, Massillo C, Rodriguez M E. Bordetella pertussis entry into respiratory epithelial cells and intracellular survival. Pathog Dis. December 2013; 69(3):194-204. doi: 10.1111/2049-632X.12072
23. Ishibashi Y, Relman D A, Nishikawa A. Invasion of human respiratory epithelial cells by Bordetella pertussis: possible role for a filamentous hemagglutinin Arg-Gly-Asp sequence and alpha5beta1 integrin. Microb Pathog. May 2001; 30(5):279-88. doi: 10.1006/mpat.2001.0432
24. Ewanowich C A, Sherburne R K, Man S F, Peppler M S. Bordetella parapertussis invasion of HeLa 229 cells and human respiratory epithelial cells in primary culture. Infect Immun. April 1989; 57(4):1240-7. doi: 10.1128/iai.57.4.1240-1247.1989
25. Zhang Y, Tam J W, Mena P, van der Velden A W, Bliska J B. CCR2+ Inflammatory Dendritic Cells and Translocation of Antigen by Type III Secretion Are Required for the Exceptionally Large CD8+ T Cell Response to the Protective YopE69-77 Epitope during Yersinia Infection. PLoS Pathog. October 2015; 11(10):e1005167. doi: 10.1371/journal.ppat. 1005167
26. Akitsu A, Ishigame H, Kakuta S, et al. IL-1 receptor antagonist-deficient mice develop autoimmune arthritis due to intrinsic activation of IL-17-producing CCR2(+)Vγ6(+)γδ T cells. Nat Commun. Jun. 25, 2015; 6:7464. doi: 10.1038/ncomms8464
27. Belcher T, Dubois V, Rivera-Millot A, Locht C, Jacob-Dubuisson F. Pathogenicity and virulence of. Virulence. December 2021; 12(1):2608-2632. doi: 10.1080/21505594.2021.1980987
28. Locht C, Antoine R. The History of Pertussis Toxin. Toxins (Basel). Sep. 5, 2021; 13(9) doi: 10.3390/toxins13090623
29. Carbonetti N H. Pertussis toxin and adenylate cyclase toxin: key virulence factors of Bordetella pertussis and cell biology tools. Future Microbiol. March 2010; 5(3):455-69. doi: 10.2217/fmb.09.133
30. Ladant D. Bioengineering of. Toxins (Basel). Jan. 22, 2021; 13(2) doi: 10.3390/toxins13020083
31. Moon K, Bonocora R P, Kim D D, et al. The BvgAS Regulon of. MBio. October 2017; 8(5) doi: 10.1128/mBio.01526-17
32. Petráčková D, Farman M R, Amman F, et al. Transcriptional profiling of human macrophages during infection with. RNA Biol. May 2020; 17(5):731-742. doi: 10.1080/15476286.2020.1727694
33. Coutte L, Antoine R, Slupek S, et al. Combined RNAseq and ChIPseq Analyses of the BvgA Virulence Regulator of Bordetella pertussis. mSystems. May 19, 2020; 5(3) doi: 10.1128/mSystems.00208-20
34. Gestal M C, Howard L K, Dewan K K, Harvill E T. Bbvac: A Live Vaccine Candidate That Provides Long-Lasting Anamnestic and Th17-Mediated Immunity against the Three Classical. mSphere. Feb. 23, 2022; 7(1):e0089221. doi: 10.1128/msphere.00892-21
35. Ochkur S I, Doyle A D, Jacobsen E A, et al. Frontline Science: Eosinophil-deficient MBP-1 and EPX double-knockout mice link pulmonary remodeling and airway dysfunction with type 2 inflammation. J Leukoc Biol. September 2017; 102(3):589-599. doi: 10.1189/jlb.3HI1116-488RR
36. Arnold I C, Artola-Borán M, Tallón de Lara P, et al. Eosinophils suppress Th1 responses and restrict bacterially induced gastrointestinal inflammation. J Exp Med. August 2018; 215(8):2055-2072. doi: 10.1084/jem.20172049
37. Mai E, Limkar A R, Percopo C M, Rosenberg H F. Generation of Mouse Eosinophils in Tissue Culture from Unselected Bone Marrow Progenitors. Methods Mol Biol. 2021; 2241:37-47. doi: 10.1007/978-1-0716-1095-4_4
38. Sugawara R, Lee E J, Jang M S, et al. Small intestinal eosinophils regulate Th17 cells by producing IL-1 receptor antagonist. J Exp Med. Apr. 4, 2016; 213(4):555-67. doi: 10.1084/jem.20141388
39. Ondari E, Calvino-Sanles E, First N J, Gestal M C. Eosinophils and Bacteria, the Beginning of a Story. Int J Mol Sci. Jul. 27, 2021; 22(15) doi: 10.3390/ijms22158004
40. Irikura V M, Hirsch E, Hirsh D. Effects of interleukin-1 receptor antagonist overexpression on infection by Listeria monocytogenes. Infect Immun. April 1999; 67(4):1901-9. doi: 10.1128/IAI.67.4.1901-1909.1999
41. Ali A, Na M, Svensson M N, et al. IL-1 Receptor Antagonist Treatment Aggravates Staphylococcal Septic Arthritis and Sepsis in Mice. PLoS One. 2015; 10(7):e0131645. doi: 10.1371/journal.pone.0131645
42. Iannitti R G, Napolioni V, Oikonomou V, et al. IL-1 receptor antagonist ameliorates inflammasome-dependent inflammation in murine and human cystic fibrosis. Nat Commun. Mar. 14, 2016; 7:10791. doi: 10.1038/ncomms10791
43. Zuverink M, Barbieri J T. From GFP to β-lactamase: advancing intact cell imaging for toxins and effectors. Pathog Dis. December 2015; 73(9): ftv097. doi: 10.1093/femspd/ftv097
44. Kroes M M, Miranda-Bedate A, Jacobi R H J, et al. Bordetella pertussis-infected innate immune cells drive the anti-pertussis response of human airway epithelium. Sci Rep. Mar. 7, 2022; 12(1):3622. doi: 10.1038/s41598-022-07603-8
45. Chung L K, Park Y H, Zheng Y, et al. The Yersinia Virulence Factor YopM Hijacks Host Kinases to Inhibit Type III Effector-Triggered Activation of the Pyrin Inflammasome. Cell Host Microbe. Sep. 14, 2016; 20(3):296-306. doi: 10.1016/j.chom.2016.07.018
46. Medici N P, Bliska J B. Methods for Detection of Pyrin Inflammasome Assembly in Macrophages Infected with Yersinia spp. Methods Mol Biol. 2019; 2010:241-255. doi: 10.1007/978-1-4939-9541-7_17
47. Yahalom A, Davidov G, Kolusheva S, et al. Structure and membrane-targeting of a Bordetella pertussis effector N-terminal domain. Biochim Biophys Acta Biomembr. Dec. 1, 2019; 1861(12):183054. doi: 10.1016/j.bbamem.2019.183054
48. Mattoo S, Yuk M H, Huang L L, Miller J F. Regulation of type III secretion in Bordetella. Mol Microbiol. May 2004; 52(4):1201-14. doi: 10.1111/j.1365-2958.2004.04053.x
49. Mattoo S, Miller J F, Cotter P A. Role of Bordetella bronchiseptica fimbriae in tracheal colonization and development of a humoral immune response. Infect Immun. April 2000; 68(4):2024-33. doi: 10.1128/iai.68.4.2024-2033.2000
50. Sengupta R, Poderycki M J, Mattoo S. CryoAPEX—an electron tomography tool for subcellular localization of membrane proteins. J Cell Sci. Mar. 18, 2019; 132(6)doi: 10.1242/jcs.222315
51. Mattoo S, Durrant E, Chen M J, et al. Comparative analysis of Histophilus somni immunoglobulin-binding protein A (IbpA) with other fic domain-containing enzymes reveals differences in substrate and nucleotide specificities. J Biol Chem. Sep. 16, 2011; 286(37):32834-42. doi: 10.1074/jbc.M111.227603
52. Li B, Smith T J. PI3K/AKT pathway mediates induction of IL-1R A by TSH in fibrocytes: modulation by PTEN. J Clin Endocrinol Metab. September 2014; 99(9):3363-72. doi: 10.1210/jc.2014-1257
53. Lam S S, Martell J D, Kamer K J, et al. Directed evolution of APEX2 for electron microscopy and proximity labeling. Nat Methods. January 2015; 12(1):51-4. doi: 10.1038/nmeth.3179
54. Sanyal A, Zbornik E A, Watson B G, et al. Kinetic and structural parameters governing Fic-mediated adenylylation/AMPylation of the Hsp70 chaperone, BiP/GRP78. Cell Stress Chaperones. July 2021; 26(4):639-656. doi: 10.1007/s12192-021-01208-2

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

The invention claimed is:

1. A method of treating or preventing and/or treating a bacterial infection in a subject, the method comprising administering to a subject an IL-1RA inhibitor.

2. The method of claim 1, wherein the bacteria comprises Bordetella spp.

3. The method of claim 2, wherein the bacteria comprises B. pertussis, B. parapertussis, B. bronchiseptica, B. holmesii, B. hinzii, B. pseudohinzii, B. ansorpii, B. trematum, B. avium, B. petrii, or any combination thereof.

4. The method of claim 1, wherein the bacteria comprises an antibiotic resistant bacteria or a non-resistant bacteria.

5. The method of claim 1, wherein the bacteria comprises a type 3 secretion system.

6. The method of claim 1, wherein the infection comprises an acute infection or a chronic infection.

7. The method of claim 1, wherein the infection comprises a respiratory infection or a non-respiratory infection.

8. The method of claim 1, wherein the IL-1RA inhibitor comprises an anti-IL-1RA antibody.

9. The method of claim 8, wherein the anti-IL-1RA antibody comprises a monoclonal antibody.

10. The method of claim 1, wherein the IL-1RA inhibitor comprises a small molecule.

11. The method of claim 1, further comprising administering to the subject an antibiotic.

12. The method of claim 11, wherein the antibiotic comprises an antibacterial agent, an antifungal agent, and/or an antiviral agent.

13. The method of claim 1, wherein the IL-1RA inhibitor is administered intranasally, intramuscular, oral or intraperitoneally.

14. A method of sensitizing an antibiotic resistant bacterial infection in a subject to an antibiotic, the method comprising administering to the subject an IL-1RA inhibitor.

15. The method of claim 14, wherein the bacteria comprises Bordetella spp.

16. The method of claim 15, wherein the bacteria comprises B. pertussis, B. parapertussis, B. bronchiseptica, B. holmesii, B. hinzii, B. pseudohinzii, B. ansorpii, B. trematum, B. avium, B. petrii, or any combination thereof.

17. The method of claim 14, wherein the bacteria comprises a type 3 secretion system.

18. The method of claim 14, wherein the infection comprises an acute infection or a chronic infection.

19. The method of claim 14, wherein the infection comprises a respiratory infection or a non-respiratory infection.

20. The method of claim 14, wherein the IL-1RA inhibitor comprises an anti-IL-1RA antibody.

21. The method of claim 20, wherein the anti-IL-1RA antibody comprises a monoclonal antibody.

22. The method of claim 14, wherein the IL-1RA inhibitor comprises a small molecule.

23. The method of claim 14, further comprising administering to the subject an antibiotic.

24. The method of claim 23, wherein the antibiotic comprises an antibacterial agent, an antifungal agent, and/or an antiviral agent

25. The method of claim 14, wherein the IL-1RA inhibitor is administered intranasally, intramuscular, oral or intraperitoneally.
